(19) United States
(12) Patent Application Publication
(10) Pub. No.: US 2006/0172992 A1
(43) Pub. Date: Aug. 3, 2006

(54) Therapeutic agent for overactive bladder resulting from cerebral infarction

(75) Inventors: Osamu Yokoyama, Fukui (JP); Masaharu Nakai, Fukui (JP)

Correspondence Address: DICKSTEIN SHAPIRO MORIN & OSHINSKY LLP, 1177 AVENUE OF THE AMERICAS (6TH AVENUE), 41 ST FL., NEW YORK, NY, 10036-2714, US

(73) Assignee: EISAI CO., LTD., Tokyo (JP)

(21) Appl. No.: 11/203,901

(22) Filed: Aug. 15, 2005

Related U.S. Application Data
(60) Provisional application No. 60/601,425, filed on Aug. 13, 2004.

(51) Int. Cl. A61K 31/55; A61K 31/445; A61K 31/473; A61K 31/407

(52) U.S. Cl. 514/214.01; 514/319; 514/297; 514/411

(57) ABSTRACT

An agent for treating overactive bladder resulting from cerebral infarction, comprising administrating a compound having a cholinesterase inhibitory activity or a pharmacologically acceptable salt thereof.

### FIELD OF THE INVENTION

The present invention relates to an agent and a method for treating overactive bladder resulting from cerebral infarction.

### BACKGROUND OF THE INVENTION

Overactive bladder is a disease recently recognized by the International Continence Society (ICS), whose major symptom being urinary urgency, which may involve urinary frequency, sometimes causing urinary incontinence. Drugs that can be used by urologists at present for treating overactive bladder such as urinary urgency, urinary frequency and urinary incontinence are limited to anticholinergic agents (antimuscarinic agents). While anticholinergic agents suppress bladder contractions via acetylcholine (ACh), they are also associated with common side effects such as dry mouth (salivation disorder) and constipation. This is because a subtype of muscarine receptor (M3) in the bladder commonly exist in the salivary gland and the gastrointestinal tract. Therefore, patients with gastrointestinal tract obstruction (such as ileus) cannot be administered with an anticholinergic agent.

Overactive bladders are observed in 50-70% of the patients with lower urinary tract obstruction such as prostatic hyperplasia, and administration of anticholinergic agents may worsen their drainage obstruction. Furthermore, anticholinergic agents are mentioned of its transfer to the nerve center where it may possibly damage higher brain functions (recognition, learning, emotion, memory and sleep). From this viewpoint, drugs that rely on new mechanism have been expected.

Overactive bladder is found in many patients suffering from brain diseases such as cerebral infarction, cerebral hemorrhage and Parkinson's disease. In addition, it is reported that ischemia in the brain is associated with deterioration of acetylcholine (ACh) system functions. So far, we have studied how this deterioration of the ACh nerve function is related with overactive bladder. As a result, we came to consider that ACh system that projects to the cerebral cortex from the forebrain basal ganglia projects suppressively to the micturition reflex center, and since this projecting system is antagonized by pirenzepine, i.e., a muscarine M1 receptor blocking agent, overactive bladder is mediated by muscarine M1 receptor (Yokoyama O, Ootsuka N, Komatsu K, Kodama K, Yotsuyanagi S, Niikura S, Nagasaka Y, Nakada Y, Kanie S, Namiki M: Forebrain muscarinic control of micturition reflex in rats. Neuropharmocology 41:629-638, 2001). When Aniracetam that stimulates ACh release in the brain is administered to a rat or human with overactive bladder caused by cerebrovascular disease, suppression of micturition reflex is observed (Nakada Y, Yokoyama O, Kamatsu K, Kodama K, Yotsuyanagi S, Niikura S, Nagasaka Y and Namiki M: Effects of aniracetam on bladder overactivity in rats with cerebral infarction. J Pharmacol Exp Ther 293: 921-928, 2000, and Osamu Yokoyama: Micturition Disorder, From Basic Research to Clinical Application, Journal of Japanese Urological Association 91: 140, 2000), which suggests that activation of ACh system in the brain may possibly ameliorate overactive bladder.

On the other hand, as therapeutic agents for lower urinary tract disorder, several compounds with acetylcholinesterase inhibitory activities have been reported. Lower urinary tract disease can be classified into micturition disorders and urine collection disorders. As one of the therapeutic agents for the former disorders, a non-carbamate amine compound with an acetylcholinesterase inhibitory activity has been reported (International Patent Publication No. 00/18391 pamphlet). However, as to the latter case, i.e., urine collection disorders involved in the overactive bladder such as urinary urgency, urinary frequency and urinary incontinence, no disclosure or suggestion has been made.

Donepezil hydrochloride is a substance that reversibly inhibits acetylcholinesterase, i.e., an acetylcholine-degrading enzyme, which increases the amount of acetylcholine in the brain and activates cholinergic nervous system in the brain. This substance is extensively used as therapeutic agents for senile dementia of Alzheimer type and Alzheimer's disease (Japanese Patent No. 2578475). However, whether this centrally-active acetylcholinesterase inhibitor, donepezil hydrochloride, has effect on urine collection disorder associated with overactive bladder such as urinary urgency, urinary frequency and urinary incontinence resulting from cerebral infarction has not been confirmed.

### SUMMARY OF THE INVENTION

The present invention provides a drug effective in treating urine collection disorder associated with overactive bladder having symptoms such as urinary urgency, urinary frequency and urinary incontinence.

As a result of devoting studies on the above-described problems, we found that when donepezil hydrochloride that inhibits acetylcholinesterase in the brain and that increases acetylcholine (ACh) in the brain was administered to rats with cerebral infarction, micturition reflex was suppressed, i.e., overactive bladder was ameliorated (Masaharu Nakai et al., Journal of Neurogenic Bladder Society 14:172, 2003 (Abstracts), and Masaharu Nakai et al., Journal of Japanese Urological Association 95: 413, 2004 (Abstracts)). This result suggests that donepezil hydrochloride activates ACh system in the brain and possibly ameliorates overactive bladder resulting from cerebral infarction. Based on these findings, we completed the present invention.

Thus, the present invention provides the followings.

(1) A method for treating overactive bladder resulting from cerebral infarction, comprising administrating a compound having a cholinesterase inhibitory activity, a pharmacologically acceptable salt or a solvate thereof to a patient with the overactive bladder resulting from cerebral infarction.

An example of a compound having a cholinesterase inhibitory activity used in the method of the present invention includes a cyclic amine derivatives represented by the following general formula: 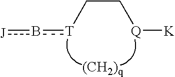
(wherein, J is:

(a) a substituted or unsubstituted (1) phenyl group, (2) pyridyl group, (3) pyradyl group, (4) quinolyl group, (5) cyclohexyl group, (6) quinoxalyl group or (7) furyl group;

(b) a monovalent or divalent group derived from a group selected from the group consisting of (1) indanyl, (2) indanonyl, (3) indenyl, (4) indenonyl, (5) indandionyl, (6) tetralonyl, (7) benzsuberonyl, (8) indanolyl, or (9) a group represented by formula 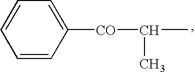
in all of which a phenyl group may be substituted;

(c) a monovalent group derived from a cyclic amide compound;

(d) a lower alkyl group; or

(e) a group represented by formula R¹—CH═CH— (wherein R¹ is a hydrogen atom or a lower alkoxycarbonyl group),

B is a group represented by formula 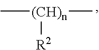
a group represented by formula 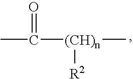
a group represented by formula 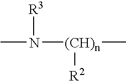
(wherein, R³ is a hydrogen atom, a lower alkyl group, an acyl group, a lower alkylsulfonyl group, a substituted or unsubstituted phenyl group or a benzyl group), a group represented by formula 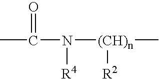
(wherein, R⁴ is a hydrogen atom, a lower alkyl group or a phenyl group), a group represented by formula 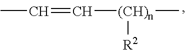
a group represented by formula 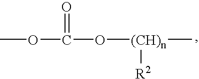
a group represented by formula 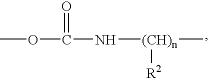
a group represented by formula 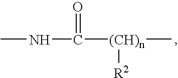
a group represented by formula 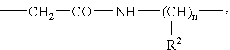
a group represented by formula 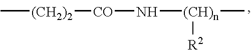
a group represented by formula 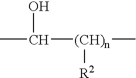
(wherein, n is 0 or an integer of 1 to 10, and R² is a hydrogen atom or a methyl group), a group represented by formula ═(CH—CH═CH)_{b}— (wherein, b is an integer of 1 to 3), a group represented by formula ═CH—(CH₂)_{c}— (wherein, c is 0 or an integer of 1 to 9), a group represented by formula ═(CH—CH)_{d}═ (wherein, d is 0 or an integer of 1 to 5), a group represented by formula 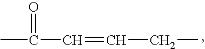
a group represented by formula 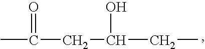
a group represented by formula 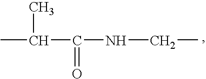
a group represented by formula 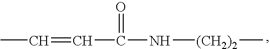
a group represented by formula —NH—, a group represented by formula —O—, a group represented by formula —S—, a dialkylaminoalkylcarbonyl group or a lower alkoxycarbonyl group,

T is a nitrogen atom or a carbon atom,

Q is a nitrogen atom, a carbon atom or a group represented by formula

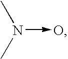

K is a hydrogen atom, a substituted or unsubstituted phenyl group, an arylalkyl group in which a phenyl group may be substituted, a cinnamyl group in which a phenyl group may be substituted, a lower alkyl group, a pyridylmethyl group, a cycloalkylalkyl group, an adamantanemethyl group, a furylmethyl group, a substituted or unsubstituted cycloalkyl group, a lower alkoxycarbonyl group or an acyl group,

q is an integer of 1 to 3, and

[symbol] indicates a single bond or a double bond).

Specifically, said J may be a group selected from the group consisting of substituted or unsubstituted (1) phenyl group, (2) pyridyl group, (3) pyradyl group, (4) quinolyl group, (5) cyclohexyl group, (6) quinoxalyl group and (7) furyl group. Furthermore, said J may be a monovalent group derived from a cyclic amide compound.

The compound having a cholinesterase inhibitory activity described above may be a cyclic amine derivative represented by the following general formula: 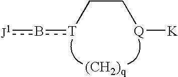
(wherein, J¹ is a monovalent or divalent group derived from a group selected from the following group consisting of (1) indanyl, (2) indanonyl, (3) indenyl, (4) indenonyl, (5) indandionyl, (6) tetralonyl, (7) benzsuberonyl, (8) indanolyl and (9) a group represented by formula 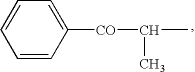
in all of which a phenyl group may be substituted,

B is a group represented by formula 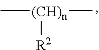
a group represented by formula 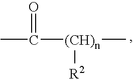
a group represented by formula 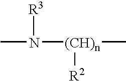
(wherein, R³ is a hydrogen atom, a lower alkyl group, an acyl group, a lower alkylsulfonyl group, a substituted or unsubstituted phenyl group or a benzyl group), a group represented by formula 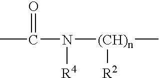
(wherein, R⁴ is a hydrogen atom, a lower alkyl group a phenyl group), a group represented by formula 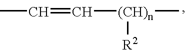
a group represented by formula 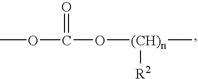
a group represented by formula 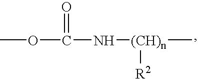
a group represented by 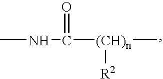
a group represented by formula 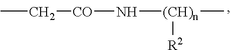
a group represented by formula 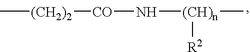
a group represented by formula 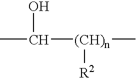
(wherein, n is 0 or an integer of 1 to 10, and R² is a hydrogen atom or a methyl group), a group represented by formula ═(CH—CH═CH)_{b}— (wherein, b is an integer of 1 to 3), a group represented by formula ═CH—(CH₂)_{c}— (wherein, c is 0 or an integer of 1 to 9), a group represented by formula ═(CH—CH)_{d}═ (wherein, d is 0 or an integer of 1 to 5), a group represented by formula 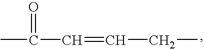
a group represented by formula 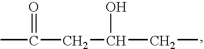
a group represented by formula 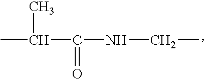
a group represented by formula 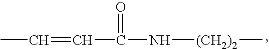
a group represented by formula —NH—, a group represented by formula —O—, a group represented by formula —S—, a dialkylaminoalkylcarbonyl group or a lower alkoxycarbonyl group,

T is a nitrogen atom or a carbon atom,

Q is a nitrogen atom, a carbon atom or a group represented by formula

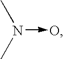

K is a hydrogen atom, a substituted or unsubstituted phenyl group, an arylalkyl group in which a phenyl group may be substituted, a cinnamyl group in which a phenyl group may be substituted, a lower alkyl group, a pyridylmethyl group, a cycloalkylalkyl group, an adamantanemethyl group, a furylmethyl group, a substituted or unsubstituted cycloalkyl group, a lower alkoxycarbonyl group or an acyl group,

q is an integer of 1 to 3, and

[symbol] indicates a single bond or a double bond).

Specifically, B may be a group represented by formula 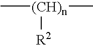
(wherein, n is 0 or an integer of 1 to 10 and R² is a hydrogen atom or a methyl group), a group represented by formula —CH═CH—(CH)ₙR²— (wherein, n is 0 or an integer of 1 to 10 and R² is a hydrogen atom or a methyl group), a group represented by formula ═(CH—CH═CH)_{b}— (wherein, b is an integer of 1 to 3), a group represented by formula ═CH—(CH₂)_{c}— (wherein, c is 0 or an integer of 1 to 9) or a group represented by formula ═(CH—CH)_{d}═ (wherein, d is 0 or an integer of 1 to 5).

Furthermore, the compound having the cholinesterase inhibitory activity described above may be a cyclic amine derivative represented by the following general formula: 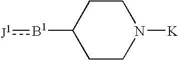
(wherein, J¹ is a monovalent or divalent group derived from a group selected from the group consisting of (1) indanyl, (2) indanonyl, (3) indenyl, (4) indenonyl, (5) indandionyl, (6) tetralonyl, (7) benzsuberonyl, (8) indanolyl and (9) a group represented by formula 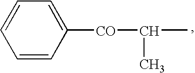
in all of which a phenyl group may be substituted,

B¹ is a group represented by formula 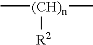
(wherein, n is 0 or an integer of 1 to 10, and R² is a hydrogen atom or a methyl group), a group represented by formula —CH═CH—(CH)ₙR²— (wherein, n is 0 or an integer of 1 to 10 and R² is a hydrogen atom or a methyl group), a group represented by formula ═(CH—CH═CH)_{b}— (wherein, b is an integer of 1 to 3), a group represented by formula ═CH—(CH₂)ₙ— (wherein, c is 0 or an integer of 1 to 9) or a group represented by formula ═(CH—CH)_{d}═ (wherein, d is 0 or an integer of 1 to 5), and

K is a hydrogen atom, a substituted or unsubstituted phenyl group, an arylalkyl group in which a phenyl group may be substituted, a cinnamyl group in which a phenyl group may be substituted, a lower alkyl group, a pyridylmethyl group, a cycloalkylalkyl group, an adamantanemethyl group, a furylmethyl group, a substituted or unsubstituted cycloalkyl group, a lower alkoxycarbonyl group or an acyl group).

Specifically, said K may be a substituted or unsubstituted arylalkyl group or phenyl group, and said J¹ may be a group selected from the group consisting of monovalent groups and divalent groups derived from indanonyl, indenyl and indandionyl. Furthermore, an example of J¹ includes an indanonyl group which may have as a substituent a lower alkyl group with a carbon number 1 to 6 or a lower alkoxy group with a carbon number 1 to 6.

The above-mentioned cyclic amine derivative may be at least one selected from the group consisting of: 1-benzyl-4-((5,6-dimethoxy-1-indanone)-2-yl)methylpiperidine, 1-benzyl-4-((5,6-dimethoxy-1-indanone)-2-ylidenyl)methylpiperidine, 1-benzyl-4-((5-methoxy-1-indanone)-2-yl)methylpiperidine, 1-benzyl-4-((5,6-methylenedioxy-1-indanone)-2-yl)methylpiperidine, 1-(m-nitrobenzyl)-4-((5,6-dimethoxy-1-indanone)-2-yl)methylpiperidine, 1-cyclohexylmethyl-4-((5,6-dimethoxy-1-indanone)-2-yl)methylpiperidine, 1-(m-fluorobenzyl)-4-((5,6-dimethoxy-1-indanone)-2-yl)methylpiperidine, 1-benzyl-4-(3-((5,6-dimethoxy-1-indanone)-2-yl)propyl)piperidine, 1-benzyl-4-((5-isopropoxy-6-methoxy-1-indanone)-2-yl)methylpiperidine, 1-benzyl-4-((5,6-dimethoxy-1-indanone)-2-ylidenyl)propenylpiperidine, and 1-benzyl-4-((5,6-dimethoxy-1,3-indandione)-2-yl)propenylpiperidine, or may be 1-benzyl-4-((5,6-dimethoxy-1-indanone)-2-yl)methylpiperidine. According to the present invention, a compound with a cholinesterase inhibitory activity is preferably 1-benzyl-4-((5,6-dimethoxy-1-indanone)-2-yl)methylpiperidine hydrochloride.

The compound having an acetylcholinesterase inhibitory activity described above may be galantamine, tacrine, physostigmine or rivastigmine.

(2) A process for screening a substance for suppressing overactive bladder resulting from cerebral infarction, comprising: administering a candidate substance to a non-human mammal; and detecting or determining a change in a phenotype of the overactive bladder resulting from cerebral infarction in the presence and absence of the candidate substance.

For the screening process of the present invention, the candidate substance include, for example, a compound having a cholinesterase inhibitory activity, a pharmacologically acceptable salt or a solvate thereof. Herein, the change in the phenotype of overactive bladder resulting from cerebral infarction can use as an index at least one selected from the group consisting of a bladder capacity, a bladder contraction pressure and an amount of retained urine.

### BRIEF DESCRIPTION OF THE DRAWINGS

FIG. 1 shows changes in cholineacetyltransferase activities in right and left cortices and right and left hippocampi after cerebral infarction;

FIG. 2 shows change in bladder capacities with respect to doses of intravenously injected donepezil hydrochloride;

FIG. 3 shows change in bladder capacities with respect to doses of intraventricularly injected donepezil hydrochloride;

FIG. 4 shows change in bladder capacities with respect to doses of donepezil hydrochloride;

FIG. 5 shows percentage of change in micturition contraction pressure;

FIG. 6 shows comparison of cerebral infarct volumes between a group of infarction rats administered with donepezil hydrochloride (CI+D group) and a group of cerebral infarction rats administered with vehicle (CI+Ve group);

FIG. 7 shows changes in cholineacetyltransferase activities in right and left cortices and right and left hippocampi after cerebral infarction;

FIG. 8 shows cholineacetyltransferase activity in pons after cerebral infarction;

FIG. 9 shows change in curves of pressure within bladders of rats intraventricularly administered with donepezil hydrochloride and their removed brains;

FIG. 10 shows change in bladder capacities with respect to doses of donepezil hydrochloride;

FIG. 11 shows percentage of change in micturition contraction pressure;

FIG. 12 shows change in bladder capacities upon donepezil hydrochloride administration; and

FIG. 13 shows percentage of change in micturition contraction pressure.

### DETAILED DESCRIPTION OF THE INVENTION

1. Compound with Cholinesterase (ChE) Inhibitory Activity

According to the present invention, an active substance for treating overactive bladder resulting from cerebral infarction comprises a compound with a ChE inhibitory activity, a pharmacologically acceptable salt or a solvate thereof. The compound with a ChE inhibitory activity according to the present invention refers to a substance with a ChE inhibitory activity, i.e., a substance that reversibly or irreversibly inhibits a ChE activity. In the present invention, ChE comprises acetylcholinesterase (ACHE) (EC3.1.1.7), butyrylcholinesterase or the like. Preferable features of the compound with a ChE inhibitory activity of the present invention include that it is highly selective for ACHE over butyrylcholinesterase, it effects centrally, it is capable of passing through the blood-brain barrier, and it does not cause severe side effect at a dose required for treatment.

According to the present invention, a preferable compound used as a therapeutic agent for overactive bladder resulting from cerebral infarction comprises a compound with a ChE, particularly ACHE inhibitory activity. This compound comprises a pharmacologically acceptable salt of the compound with a ChE inhibitory activity, a solvate thereof and a prodrug thereof as described below.

(1) Compound with Cholinesterase Inhibitory Activity

According to the present invention, compounds with a ChE inhibitory activity include donepezil (ARICEPT®), galantamine (Reminyl®), tacrine (Cognex®), rivastigmine (Exelon®), zifrosilone (U.S. Pat. No. 5,693,668 specification), physostigmine (Synapton) (Neurobiology of Aging 26 (2005) 939-946), ipidacrine (U.S. Pat. No. 4,550,113 specification), quilostigmine, metrifonate (Promem) (U.S. Pat. No. 4,950,658 specification), eptastigmine, velnacrine, tolserine, cymserine (U.S. Pat. No. 6,410,747 specification), mestinon, icopezil (U.S. Pat. No. 5,750,542 specification), TAK-147 (J. Med. Chem., 37(15), 2292-2299, 1994, Japanese Patent Publication No. 2650537, U.S. Pat. No. 5,273,974 specification), huperzine A (Drugs Fut., 24, 647-663, 1999), stacofylline (U.S. Pat. No. 4,599,338 specification), thiatolserine, neostigmine, eseroline, or thiacymserine, 8-[3-[1-[(3-fluorophenyl)methyl]-4-piperidinyl]-1-oxopropyl]-1,2,5,6-tetrahydro-4H-pyrrolo[3,2,1-ij]quinoline-4-one (Japanese Patent Publication No. 3512786), phenserine or ZT-1. The compound may also be a derivative or a prodrug of the above compounds. In addition, a pharmacologically acceptable salt or a solvate of the above compounds, derivatives and prodrugs may also be included as preferred embodiments of the compound with a ChE inhibitory activity. The compound with a ChE inhibitory activity also includes the compound with a ChE inhibitory activity described in International Patent Publication No. 00/18391 pamphlet.

Galantamine and derivatives thereof are described in U.S. Pat. No. 4,663,318 specification, International Patent Publication No. 88/08708 pamphlet, International Patent Publication No. 97/03987 pamphlet, U.S. Pat. No. 6,316,439 specification, U.S. Pat. No. 6,323,195 specification, U.S. Pat. No. 6,323,196 specification and the like. Tacrine and derivatives thereof are described in U.S. Pat. No. 4,631,286 specification, U.S. Pat. No. 4,695,573 specification, U.S. Pat. No. 4,754,050 specification, International Patent Publication No. 88/02256 pamphlet, U.S. Pat. No. 4,835,275 specification, U.S. Pat. No. 4,839,364 specification, U.S. Pat. No. 4,999,430 specification, International Patent Publication WO97/21681 pamphlet and the like. Physostigmine and derivatives thereof are described in U.S. Pat. No. 5,077,289 specification, U.S. Pat. No. 5,177,101 specification, U.S. Pat. No. 5,302,721 specification, Japanese Laid-Open Application No. 5-306286, U.S. Pat. No. 7,166,824 specification, EP Patent No. 298202 specification, International Patent Publication No. 98/27096 pamphlet, J. Pharm. Exp. Therap., 249 (1), 194-202, 1989 and the like. Rivastigmine and derivatives thereof are described in EP Patent No. 193926 specification, International Patent Publication No. 98/26775 pamphlet, International Patent Publication No. 98/27055 pamphlet and the like.

“Prodrug” as used herein means a drug obtained by chemically modifying “an active ingredient of a drug” (i.e., a “drug” corresponding to the prodrug) into an inactive substance for the purpose of bioavailability improvement, alleviation of side effects or the like, which, after absorption, is metabolized to an active ingredient in the body and exerts action. Thus, the term “prodrug” refers to any compound that has a lower intrinsic activity than a corresponding “drug” but which, when administered to a biological system, generates the “drug” substance as a result of spontaneous chemical reaction, enzyme catalysis or metabolic reaction. Examples of such prodrugs include those in which an amino group, a hydroxyl group or a carboxyl group of the above-exemplified compound or a compound represented by the general formula below has been acylated, alkylated, phosphorylated, borated, carbonated, esterified, amidated or urethanated. This exemplified group, however, merely represents typical examples and thus is not comprehensive. Those skilled in the art can prepare other various known prodrugs from the above-exemplified compound or the compound represented by the general formula below according to a known method. A prodrug comprising the above-exemplified compound or the compound represented by the general formula below is within the scope of the invention.

(2) Cyclic Amine Derivatives

According to the present invention, preferred examples of a compound with a ChE inhibitory activity, specifically an ACHE inhibitory activity further include a cyclic amine derivative represented by the following general formula (I), a pharmacologically acceptable salt and a solvate thereof. According to the present invention, a compound with a ChE inhibitory activity is preferably 1-benzyl-4-[(5,6-dimethoxy-1-indanone)-2-yl]methylpiperidine (donepezil), a pharmacologically acceptable salt or a solvate thereof, more preferably 1-benzyl-4-[(5,6-dimethoxy-1-indanone)-2-yl]methylpiperidine hydrochloride (donepezil hydrochloride), i.e., ARICEPT®.

General Formula (I) 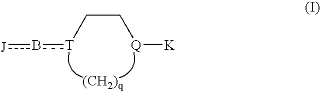
(wherein, J refers to one selected from groups (a) to (e) listed below:

(a) a substituted or unsubstituted (1) phenyl group, (2) pyridyl group, (3) pyradyl group, (4) quinolyl group, (5) cyclohexyl group, (6) quinoxalyl group or (7) furyl group;

(b) a monovalent or divalent group derived from one selected from the group consisting of (1) indanyl, (2) indanonyl, (3) indenyl, (4) indenonyl, (5) indandionyl, (6) tetralonyl, (7) benzsuberonyl, (8) indanolyl, and (9) a group represented by formula 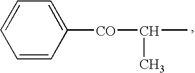
in all of which a phenyl group may be substituted,

(c) a monovalent group derived from a cyclic amide compound,

(d) a lower alkyl group, or

(e) a group represented by formula R¹—CH═CH— (wherein, R¹ is a hydrogen atom or a lower alkoxycarbonyl group),

B refers to a group represented by formula 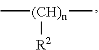
a group represented by formula 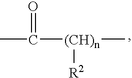
a group represented by formula 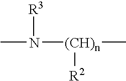
(wherein, R³ is a hydrogen atom, a lower alkyl group, an acyl group, a lower alkylsulfonyl group, a substituted or unsubstituted phenyl group or a benzyl group), a group represented by formula 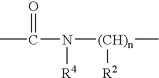
(wherein, R⁴ is a hydrogen atom, a lower alkyl group or a phenyl group), a group represented by formula 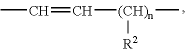
a group represented by formula 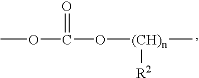
a group represented by formula 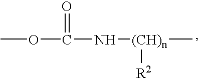
a group represented by formula 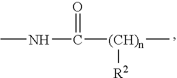
a group represented by formula 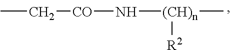
a group represented by formula 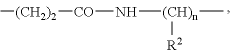
a group represented by formula 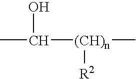
(wherein, n is 0 or an integer of 1 to 10, and R² is a hydrogen atom or a methyl group), a group represented by formula ═(CH—CH═CH)_{b}— (wherein, b is an integer of 1 to 3), a group represented by formula ═CH—(CH₂)_{c}— (wherein, c is 0 or an integer of 1 to 9), a group represented by formula ═(CH—CH)_{d}═ (wherein, d is 0 or an integer of 1 to 5), a group represented by formula 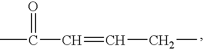
a group represented by formula 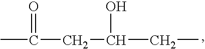
a group represented by formula 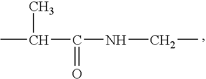
a group represented by formula 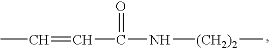
a group represented by formula —NH—, a group represented by formula —O—, a group represented by formula —S—, a dialkylaminoalkylcarbonyl group or a lower alkoxycarbonyl group,

T represents a nitrogen atom or a carbon atom,

Q represents a nitrogen atom, a carbon atom or a group represented by formula

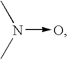

K is a hydrogen atom, a substituted or unsubstituted phenyl group, an arylalkyl group in which a phenyl group may be substituted, a cinnamyl group in which a phenyl group may be substituted, a lower alkyl group, a pyridylmethyl group, a cycloalkylalkyl group, an adamantanemethyl group, a furylmethyl group, a substituted or unsubstituted cycloalkyl group, a lower alkoxycarbonyl group or an acyl group,

q is an integer of 1 to 3, and

[symbol] indicates a single bond or a double bond).

“A lower alkyl group” as used herein comprises a straight or branched alkyl group with a carbon number 1 to 6, for example, a methyl group, an ethyl group, an n-propyl group, an isopropyl group, an n-butyl group, an isobutyl group, a sec-butyl group, a tert-butyl group, a pentyl group (an amyl group), an isopentyl group, a neopentyl group, a tert-pentyl group, a 1-methylbutyl group, a 2-methylbutyl group, a 1,2-dimethylpropyl group, a hexyl group, an isohexyl group, a 1-methylpentyl group, a 2-methylpentyl group, a 3-methylpentyl group, a 1,1-dimethylbutyl group, a 1,2-dimethylbutyl group, a 2,2-dimethylbutyl group, a 1,3-dimethylbutyl group, a 2,3-dimethylbutyl group, a 3,3-dimethylbutyl group, a 1-ethylbutyl group, a 2-ethylbutyl group, a 1,1,2-trimethylpropyl group, a 1,2,2-trimethylpropyl group, a 1-ethyl-1-methylpropyl group, a 1-ethyl-2-methylpropyl group and the like. Preferable groups among them include a methyl group, an ethyl group, a propyl group and an isopropyl group, most preferable group being a methyl group. “A lower alkyl group” is described in the definition of the above compound (I) of the present invention, for example, in the definitions of J, K, R³ and R⁴.

“A lower alkoxy group” as used herein means a lower alkoxy group corresponding to the above-mentioned lower alkyl group such as a methoxy group and an ethoxy group.

“A lower alkoxycarbonyl group” as used herein means a lower alkoxycarbonyl group corresponding to the above-mentioned lower alkoxy group such as a methoxycarbonyl group, an ethoxycarbonyl group, an isopropoxycarbonyl group, an n-propoxycarbonyl group and an n-butyloxycarbonyl group.

“A cycloalkyl group” as used herein refers to a cyclic alkyl group with a carbon number 4 to 10, including but not limited to a cyclobutyl group, a cyclopentyl group and a cyclohexyl group.

“J”

In the definition of J, exemplary substituents for “(a) substituted or unsubstituted (1) phenyl group, (2) pyridyl group, (3) pyradyl group, (4) quinolyl group, (5) cyclohexyl group, (6) quinoxalyl group or (7) furyl group” include:

a lower alkyl group with a carbon number 1 to 6 such as a methyl group, an ethyl group, an n-propyl group, an isopropyl group, an n-butyl group, an isobutyl group and a tert-butyl group;

a lower alkoxy group corresponding to a lower alkyl group such as a methoxy group and an ethoxy group;

a nitro group;

a halogen such as chlorine, bromine and fluorine;

a carboxyl group;

a lower alkoxycarbonyl group corresponding to the lower alkoxy group above such as a methoxycarbonyl group, an ethoxycarbonyl group, an isopropoxycarbonyl group, an n-propoxycarbonyl group and an n-butyloxycarbonyl group;

an amino group;

a mono-lower alkylamino group;

a di-lower alkylamino group;

a carbamoyl group;

an acylamino group derived from aliphatic saturated monocarboxylic acid with a carbon number 1 to 6 such as an acetylamino group, a propionylamino group, a butyrylamino group, an isobutyrylamino group, a valerylamino group and a pivaloyl amino group;

a cycloalkyloxycarbonyl group such as a cyclohexyloxycarbonyl group;

a lower alkylaminocarbonyl group such as a methylaminocarbonyl group and an ethylaminocarbonyl group;

a lower alkylcarbonyloxy group corresponding to the lower alkyl group defined above such as a methylcarbonyloxy group, an ethylcarbonyloxy group and an n-propylcarbonyloxy group;

a halogenated lower alkyl group as represented by a trifluoromethyl group or the like;

a hydroxyl group;

a formyl group; and

a lower alkoxy lower alkyl group such as an ethoxymethyl group, a methoxymethyl group and a methoxyethyl group.

As to the above substituents, “the lower alkyl group” and “the lower alkoxy group” comprise all of the groups that can be derived from the definition described above. Groups (1) to (7) from (a) may be substituted with 1 to 3 of the same or different substituents mentioned above.

In the case of the phenyl group, the following case is also to be included in the substituted phenyl group: that is, when a group can be represented by formula 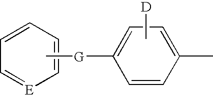
(wherein, G is a group represented by 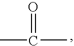
a group represented by 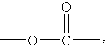
a group represented by —O—, a group represented by 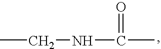
a group represented by —CH₂—O—, a group represented by —CH₂—SO₂—, a group represented by 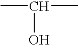
or a group represented by 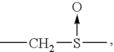
and

E represents a carbon atom or a nitrogen atom).

D may represent a lower alkyl group with a carbon number 1 to 6 such as a methyl group, an ethyl group, an n-propyl group, an isopropyl group, an n-butyl group, an isobutyl group, and a tert-butyl group;

a lower alkoxy group corresponding to the lower alkyl group above such as a methoxy group and an ethoxy group;

a nitro group;

a halogen such as chlorine, bromine and fluorine;

a carboxyl group;

a lower alkoxycarbonyl group corresponding to the lower alkoxy group above such as a methoxycarbonyl group, an ethoxycarbonyl group, an isopropoxycarbonyl group, an n-propoxycarbonyl group and an n-butyloxycarbonyl group;

an amino group;

a mono-lower amino group;

a di-lower alkylamino group;

a carbamoyl group;

an acylamino group derived from aliphatic saturated monocarboxylic acid with a carbon number 1 to 6 such as an acetylamino group, a propionylamino group, a butyrylamino group, an isobutyrylamino group, a valerylamino group and a pivaloylamino group;

a cycloalkyloxycarbonyl group such as a cyclohexyloxycarbonyl group;

a lower alkylaminocarbonyl group such as a methylaminocarbonyl group and an ethylaminocarbonyl group;

a lower alkylcarbonyloxy group corresponding to the lower alkyl group defined above such as a methylcarbonyloxy group, an ethylcarbonyloxy group and an n-propylcarbonyloxy group;

a halogenated lower alkyl group as represented by a trifluoromethyl group;

a hydroxyl group;

a formyl group;

a lower alkoxy lower alkyl group such as an ethoxymethyl group, a methoxymethyl group and a methoxyethyl group.

As to the substituents, “the lower alkyl group” and “the lower alkoxy group” comprise all of the groups that can be derived from the definition described above.

Among those mentioned above, substituents favorable for a phenyl group include a lower alkyl group, a lower alkoxy group, a nitro group, a halogenated lower alkyl group, a lower alkoxycarbonyl group, a formyl group, a hydroxyl group, a lower alkoxy lower alkyl group, a halogen, a benzoyl group and a benzylsulfonyl group. The substituents may be two or more and may be the same or different.

Preferred substituents for a pyridyl group may include a lower alkyl group, an amino group and a halogen atom.

Preferred substituents for a pyradyl group may include a lower alkoxycarbonyl group, a carboxyl group, an acylamino group, a carbamoyl group and a cycloalkyloxycarbonyl group.

When representing “J”, 2-pyridyl group, 3-pyridyl group or 4-pyridyl group is desirable as a pyridyl group, 2-pyradyl group is desirable as a pyradyl group, 2-quinolyl group or 3-quinolyl group is desirable as a quinolyl group, 2-quinoxalyl group or 3-quinoxalyl group is desirable as a quinoxalyl group, and 2-furyl group is desirable as a furyl group.

In the definition of “J”, typical examples of the monovalent or divalent group derived from (1) to (9) listed in group (b) are shown below:

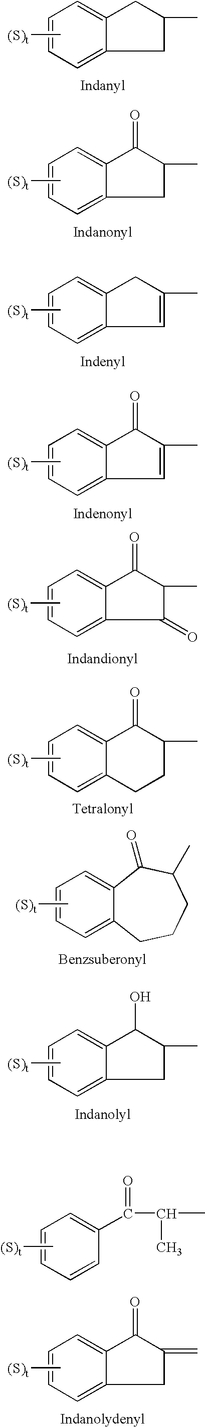

In the above series of formulae, t means 0 or an integer of 1 to 4, indicating that the phenyl group is substituted by 0 to 4 groups indicated by S which may be the same or different. S identically or differently indicates one of the substituents listed in (a) in the definition of J or a hydrogen atom and preferably includes a hydrogen atom (unsubstituted), a lower alkyl group or a lower alkoxy group. Furthermore, the phenyl group may be substituted by an alkylenedioxy group such as a methylenedioxy group or an ethylenedioxy group between adjacent carbons of the phenyl ring.

Among those mentioned above, a preferable case is where no substitution exist, where 1 to 3 methoxy groups or isopropoxy groups are substituted, or where a methylenedioxy group is substituted. Most preferable case is where no substitution exist, or where 1 to 3 methoxy groups are substituted.

The above-mentioned indanolydenyl is an example where a divalent group in which a phenyl group listed in (b) in the definition of J may be substituted, i.e., a typical divalent group derived from (2) indanonyl in J (b).

In the definition of J, examples of the monovalent group derived from a cyclic amide compound from (c) include, for example, quinazolone, tetrahydroisoquinoline-one, tetrahydrobenzodiazepine-one and hexahydrobenzazocin-one, but are not limited thereto as long as a cyclic amide exists in the structural formula.

The cyclic amide may be derived from a monocyclic ring or a condensed heterocyclic ring. Preferably, the condensed heterocyclic ring is a condensed heterocyclic ring with a phenyl ring. In this case, the phenyl ring may be substituted with a lower alkyl group with a carbon number 1 to 6, preferably a methyl group, a lower alkoxy group with a carbon number 1 to 6, preferably a methoxy group or a halogen atom.

Preferable examples include the following: 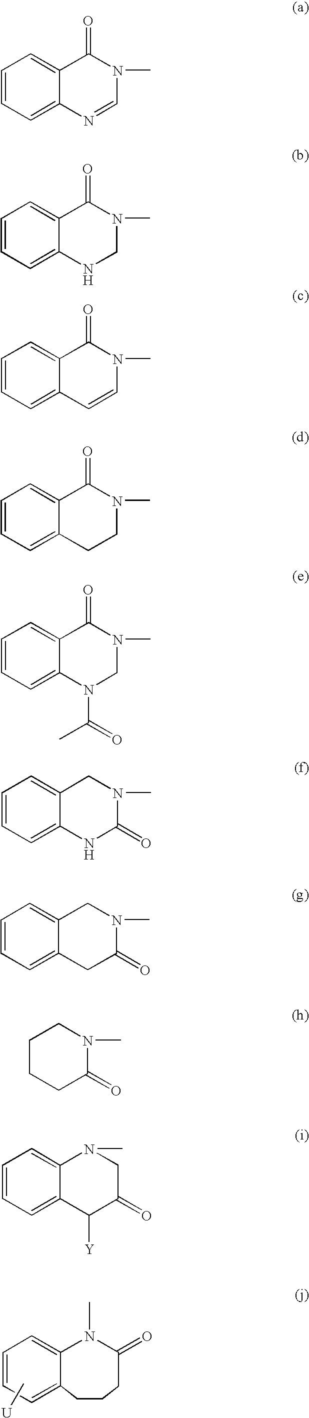 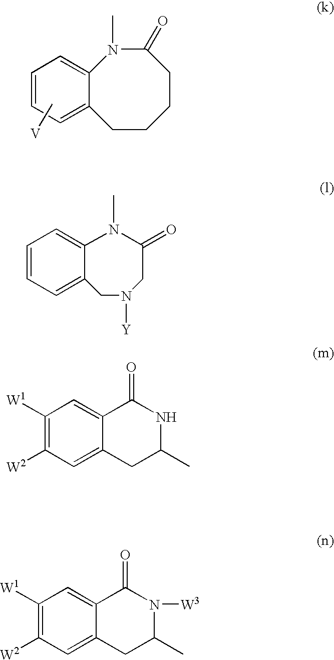
(wherein, Y in formulae (i) and (l) represents a hydrogen atom or a lower alkyl group, V in formula (k) represents a hydrogen atom or a lower alkoxy group, W¹ and W² in formulae (m) and (n) each independently represent, identically or differently, a hydrogen atom, a lower alkyl group or a lower alkoxy group, and W³ is a hydrogen atom or a lower alkyl group. U in formula (j) represents a hydrogen atom, a lower alkyl group or a lower alkoxy group.

The rings on the right side in formulae (j) and (l) are seven-membered rings, and the ring on the right side in formula (k) is an eight-membered ring.

For the definition of J, “(d) a lower alkyl group” is as described above.

Among those included in the above definition of J, groups included in (a) to (c) are preferable, most preferable group being a monovalent group derived from indanone (indanonyl) included in (b) where a phenyl ring may be substituted or unsubstituted, and a monovalent group derived from a cyclic amide compound included in (c).

“B”

For the definition of B, a group represented by formula: 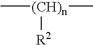
is indicated as formula —(CH₂)ₙ— when R² is a hydrogen atom. In this case, any of the carbon atoms of the alkylene chain may further bind to one or more methyl groups and n is preferably 1 to 3.

In B, examples of “dialkylaminoalkylcarbonyl group” include, for example, N,N-dimethylaminoalkylcarbonyl group, N,N-diethylaminoalkylcarbonyl group, N,N-diisopropylaminoalkylcarbonyl group, and N-methyl-N-ethylaminoalkylcarbonyl group.

As to a series of groups of B, a group including an amide group is also preferable.

Examples of preferable groups further include a group represented by formula —CH═CH—(CH)ₙR²— (wherein, n is 0 or an integer of 1 to 10, and R² is a hydrogen atom or a methyl group), a group represented by formula ═(CH—CH═CH)_{b}— (wherein, b is an integer of 1 to 3), a group represented by formula ═CH—(CH₂)_{c}— (wherein, c is 0 or an integer of 1 to 9), a group represented by formula ═(CH—CH)_{d}═ (wherein, d represents 0 or an integer of 1 to 5), a group represented by formula —NH—, a group represented by formula —O— and a group represented by formula —S—.

“T”, “Q” and “q”

A ring 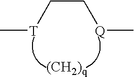
may be a five- to seven-membered ring. Specifically, examples of such ring include 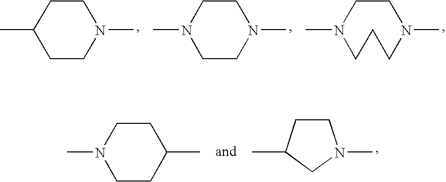
although particularly preferable ring is piperidine represented by formula

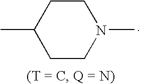

“K” and “Bonds”

As to expressions “substituted or unsubstituted phenyl group”, “substituted or unsubstituted arylalkyl group (where a phenyl group may be substituted)”, “cinnamyl group where a phenyl group may be substituted”, and “cycloalkyl group which may be substituted” in the definition of K, the substituents are the same as those defined in definition of J for (a) (1) to (7). These are preferably unsubstituted or may be substituted with a nitro group, a lower alkyl group such as methyl or a halogen such as fluorine.

An arylalkyl group is intended to mean a benzyl group or a phenetyl group in which a phenyl ring is substituted with a substituent described above or unsubstituted.

Examples of pyridylmethyl group may specifically include 2-pyridylmethyl group, 3-pyridylmethyl group and 4-pyridylmethyl group.

As to K, an arylalkyl group where a phenyl group may be substituted, a substituted or unsubstituted phenyl group, a cinnamyl group where a phenyl group may be substituted and a cycloalkyl group which may be substituted are most preferable.

Preferable arylalkyl group is specifically, for example, a benzyl group or a phenetyl group in which a phenyl group may be substituted with a lower alkoxy group having a carbon number 1 to 6, a lower alkyl group having a carbon number 1 to 6, a hydroxyl group or the like.

[symbol] indicates a single bond or a double bond. An exemplary case of the double bond includes the above-described divalent group derived from indanone where a phenyl ring may be substituted, namely an indanolydenyl group.

Compound Group (A)

Gathering from these definitions, particularly preferable compound group include compound group (A) represented by the following general formula, i.e., a cyclic amine represented by formula: 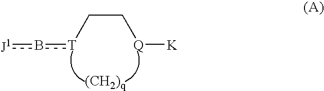
(wherein, J¹ is a monovalent or divalent group derived from a group selected from the group consisting of:
(1) indanyl,
(2) indanonyl,
(3) indenyl,
(4) indenonyl,
(5) indandionyl,
(6) tetralonyl,
(7) benzsuberonyl,
(8) indanolyl, and
(9) a group represented by formula 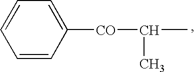
in all of which a phenyl group may be substituted; and

B, T, Q, q, K and [symbol] have the same meaning as described above), a pharmacologically acceptable salt or a solvate thereof.

In the above definition of J¹, the most preferable groups include an indanonyl group, an indandionyl group and indanolydenyl group where a phenyl group may be substituted. Specifically, a phenyl group may be unsubstituted or substituted identically or differently with a hydroxyl group, a halogen or a lower alkoxy group, and most preferably substituted with an alkylenedioxy group between adjacent carbon atoms of a phenyl ring. A lower alkoxy group refers to, for example, a methoxy group, an ethoxy group, an isopropoxy group, an n-propoxy group and an n-butoxy group with a carbon number 1 to 6, and can take a form of mono- to tetra-substitution, preferably disubstitution. Disubstitution of the methoxy group is most preferable.

Compound Group (B)

More preferable compound group included in formula (A) include a compound group represented by the following general formula (B): 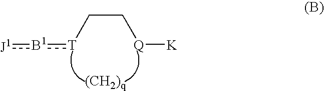
(wherein, J¹ is the same as described above,

B¹ is a group represented by 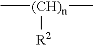
(wherein, n is 0 or an integer of 1 to 10, and R² is a hydrogen atom or a methyl group), a group represented by formula —CH═CH—(CH)R²— (wherein, n represents 0 or an integer of 1 to 10, and R² represents a hydrogen atom or a methyl group), a group represented by formula ═(CH—CH═CH)_{b}— (wherein, b is an integer of 1 to 3), a group represented by formula ═CH—(CH₂)_{c}— (wherein, c represents 0 or an integer of 1 to 9) or a group represented by formula ═(CH—CH)_{d}═ (wherein, d is 0 or an integer of 1 to 5): Preferably, B¹ is a group represented by formula —(CH)ₙR²— (wherein, n is 0 or an integer of 1 to 10, and R² is a hydrogen atom or a methyl group), more preferably —CH₂— (wherein, n=1, and R² is a hydrogen atom), or —CH₂—CH₂—CH₂— (wherein, n=3, and R² is a hydrogen atom): B¹ is preferably a group represented by formula —CH═CH—(CH)ₙR²— (wherein, n is 0 or an integer of 1 to 10, and R² is a hydrogen atom or a methyl group), more preferably —CH═CH—CH₂— (wherein, n=1 and R² is a hydrogen atom), and

T, Q, q, K and [symbol] are as described above).

Compound Group (C)

More preferable compound group included in formula (B) may include a compound group represented by the following general formula (C): 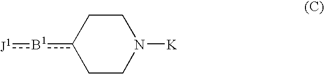
(wherein, J¹, B¹, K and [symbol] are as described above).

Specifically, the group represented by formula 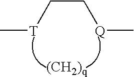
is indicated by a group represented by formula 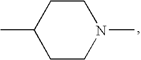
i.e., piperidine.

Compound Group (D)

More preferable compound group included in formula (C) may include a compound group represented by the following general formula (D): 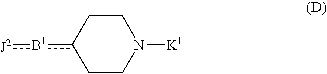
(wherein, J² is a group selected from a monovalent or divalent group derived from indanonyl where a phenyl group may be substituted (e.g., indanonyl, indanolydenyl group), indenyl and indandionyl: More preferably, J² is an indanonyl group which may have, as a substituent, a lower alkyl group with a carbon number 1 to 6 or a lower alkoxy group with a carbon number 1 to 6,

K¹ is a substituted or unsubstituted phenyl group, an arylalkyl group which may be substituted, a cinnamyl group which may be substituted or a cycloalkyl group which may be substituted, and

B¹ and [symbol] are as described above).

Moreover, a particularly preferable compound group (a compound group having a ChE inhibitory activity) of cyclic amine derivatives represented by general formula (I) or pharmacologically acceptable salts thereof includes the following:
1-benzyl-4-((5,6-dimethoxy-1-indanone)-2-yl)methylpiperidine,
1-benzyl-4-((5,6-dimethoxy-1-indanone)-2-ylidenyl)methylpiperidine,
1-benzyl-4-((5-methoxy-1-indanone)-2-yl)methylpiperidine,
1-benzyl-4-((5,6-methylenedioxy-1-indanone)-2-yl)methylpiperidine,
1-(m-nitrobenzyl)-4-((5,6-dimethoxy-1-indanone)-2-yl)methylpiperidine,
1-cyclohexylmethyl-4-((5,6-dimethoxy-1-indanone)-2-yl)methylpiperidine,
1-(m-fluorobenzyl)-4-((5,6-dimethoxy-1-indanone)-2-yl)methylpiperidine,
1-benzyl-4-(3-((5,6-dimethoxy-1-indanone)-2-yl)propyl)piperidine,
1-benzyl-4-((5-isopropoxy-6-methoxy-1-indanone)-2-yl)methylpiperidine,
1-benzyl-4-((5,6-dimethoxy-1-indanone)-2-ylidenyl)propenyl)piperidine, and
1-benzyl-4-((5,6-dimethoxy-1,3-indandione)-2-yl)propenylpiperidine,
more preferably 1-benzyl-4-((5,6-dimethoxy-1-indanone)-2-yl)methylpiperidine.

(3) Production Process

A compound having a ChE inhibitory activity, a pharmacologically acceptable salt thereof or a solvate thereof used in the present invention can be produced according to a known method. The cyclic amine derivatives represented by the general formula (I) above (e.g., donepezil hydrochloride) can readily be produced by methods disclosed, as representative examples, in Japanese Laid-Open Application No. 1-79151, Japanese Patent Publication No. 2578475, Japanese Patent Publication No. 2733203, Japanese Patent Publication No. 3078244 or U.S. Pat. No. 4,895,841. Donepezil hydrochloride is also available as a formulation such as fine granules.

Galantamine and derivatives thereof can readily be produced by methods disclosed, for example, in U.S. Pat. No. 4,663,318 specification, International Patent Publication No. 88/08708 pamphlet, International Patent Publication No. 97/03987 pamphlet, U.S. Pat. No. 6,316,439 specification, U.S. Pat. No. 6,323,195 specification and U.S. Pat. No. 6,323,196 specification.

Tacrine and derivatives thereof can readily be produced by methods disclosed, for example, in U.S. Pat. No. 4,631,286 specification, U.S. Pat. No. 4,695,573 specification, U.S. Pat. No. 4,754,050 specification, International Patent Publication No. 88/02256 pamphlet, U.S. Pat. No. 4,835,275 specification, U.S. Pat. No. 4,839,364 specification, U.S. Pat. No. 4,999,430 specification, and International Patent Publication WO97/21681 pamphlet.

Physostigmine and derivatives thereof can readily be produced by methods disclosed, for example, in U.S. Pat. No. 5,077,289 specification, U.S. Pat. No. 5,177,101 specification, U.S. Pat. No. 5,302,721 specification, Japanese Laid-Open Application No. 5-306286, U.S. Pat. No. 7,166,824 specification, EP Patent No. 298202 specification, International Patent Publication No. 98/27096 pamphlet, and J. Pharm. Exp. Therap., 249 (1), 194-202, 1989.

Rivastigmine and derivatives thereof can readily be produced by methods disclosed, for example, in EP Patent No. 193926 specification, International Patent Publication No. 98/26775 pamphlet, and International Patent Publication No. 98/27055 pamphlet.

Among these compounds, those that are commercially available can readily be obtained from, for example, chemical manufacturers.

According to the present invention, examples of pharmacologically acceptable salts include, for example, inorganic acid salts such as hydrochloride, sulfate, hydrobromate and phosphate, or organic acid salts such as formate, acetate, trifluoroacetate, maleate, tartrate, methanesulfonate, benzenesulfonate and toluenesulfonate.

In addition, depending on the choice of the substituent, for example, alkali metal salts such as sodium salt and potassium salt, alkaline earth metal salts such as calcium salt and magnesium salt, organic amine salts such as trimethylamine salt, triethylamine salt, pyridine salt, picoline salt, dicyclohexylamine salt and N,N′-dibenzylethylenediamine salt, and ammonium salt are formed.

According to the present invention, a compound having a ChE inhibitory activity or a pharmacologically acceptable salt thereof (e.g., donepezil hydrochloride) as an active ingredient for overactive bladder treatment may be an anhydride, and may form a solvate such as a hydrate. According to the present invention, a solvate is preferably a pharmacologically acceptable solvate. A pharmacologically acceptable solvate may be either a hydrate or a nonhydrate, but preferably a hydrate. A solvent such as water, alcohol (e.g., methanol, ethanol, n-propanol), dimethylformamide, dimethyl sulfoxide (DMSO) or the like may be used. For example, crystal polymorph may exist in the above-mentioned donepezil, although not limited thereto and any form of crystal may exist alone or in combination.

According to the present invention, the above-mentioned compound may have an asymmetric carbon depending on the type of substituent and may have an enantiomer, which are within the scope of the present invention.

In one specific example, if J has an indanone skeleton associated with an asymmetric carbon, a geometric isomer, an enantiomer, a diastereomer or the like may exist. All of these cases are within the scope of the present invention.

2. Therapeutic Agent for Overactive Bladder Resulting from Cerebral Infarction

According to the present invention, a therapeutic agent for overactive bladder resulting from cerebral infarction refers to a drug that increases a bladder capacity that has decreased because of cerebral infarction in human or organisms other than human such as non-human mammals including cow, monkey, avian, cat, mouse, rat, guinea pig, hamster, pig, dog and rabbit. The therapeutic agent of the present invention activates Ch nervous system in the brain by inhibiting cholinesterase (ChE) (including acetylcholinesterase (AChE)) to ameliorate overactive bladder such as urinary urgency, urinary frequency and urinary incontinence. This means that the therapeutic agent of the present invention is effective in ameliorating deterioration of overactive bladder and bladder capacity caused by deterioration of functions resulting from cerebral infarction such as deterioration of cholinergic neural action, preferably deterioration of central cholinergic neural action, or deterioration of action of choline acetyltransferase, i.e., ACh-synthesis enzyme, in the central nerve. Thus, the therapeutic agent of the present invention may effectively be used for treating overactive bladder in a patient whose cholinergic neural action has deteriorated because of cerebral infarction, for example, patients with brain disease such as cerebral infarction (e.g., lacunar infarction, atherothrombosis or cardiogenic cerebral infarction), asymptomatic cerebral infarction and cerebral hemorrhage. The therapeutic agent of the present invention desirably has no influence on micturition contraction pressure, and is not associated with urge of urination. Also, the therapeutic agent of the present invention may be termed either as a therapeutic agent or an improving agent for urinary urgency, urinary frequency, urinary incontinence and the like resulting from cerebral infarction.

The compound having a ChE inhibitory activity described above, a pharmacologically acceptable salt or a solvate thereof increases the bladder capacity. In addition, they are useful as an active ingredient of a therapeutic agent of the present invention.

Thus, the present invention also provides a method for treating overactive bladder resulting from cerebral infarction, comprising administering an effective amount of the compound having a ChE inhibitory activity described above, a pharmacologically acceptable salt or a solvate thereof to a patient.

The term “treatment” generally means an achievement of a desirable pharmacological effect and/or physiological effect. These effects can be prophylactic in terms of completely or partially preventing a disease and/or symptoms, and therapeutic in terms of partially or completely curing a disease and/or adverse effects caused by a disease. Herein, “treatment” refers to any treatment for a disease of a mammal, particularly human, and also includes general treatment as described above. “Treatment” includes, for example, the following (a) to (c):

(a) to prevent a disease or a symptom in a patient who is predisposed to the disease or the symptom but not yet diagnosed to be so;

(b) to inhibit a disease or a symptom, that is, to stop or delay the progress thereof;

(c) to alleviate a disease or a symptom, that is, to delay or eliminate the disease or the symptom, or to reverse the progress of the symptom.

A compound with a ChE inhibitory activity, a salt or a solvate thereof, or a prodrug thereof, a salt or a solvate thereof may be administered either orally or parenterally to a human or non-human mammal (e.g., intravenous injection, muscle injection, subcutaneous injection, rectal administration, transdermal administration) by any one of various means. A compound having a ChE inhibitory activity, a salt or a solvate thereof, or a prodrug thereof, a salt or a solvate thereof may be used alone or may be formulated into an appropriate formulation using a pharmaceutical carrier by employing a conventionally used method depending on the administration route.

Examples of preferable formulations include, for example, oral formulations such as tablets, powder, fine granules, granules, coated tablets, capsules, syrup and lozenge, and parenteral formulations such as inhalers, suppositories, injectable agents (including intravenous fluids), ointments, ophthalmic drops, ophthalmic ointments, nasal drops, ear drops, adhesive patches, skin pads, lotion and liposome formulations.

Examples of carriers that can be used for formulating these formulations include, for example, a generally used solvent, excipient, coating agent, binder, disintegrating agent, lubricant, colorant, flavoring or aromatic substance, and if necessary, a stabilizer, an emulsifying agent, an absorption promoter, a surfactant, a pH regulator, an antiseptics, an antioxidant, a filler, a wetting agent, a surface-active agent, a dispersant, a buffer, a preservative, a solubilizing agent, a suspending agent, a thickening agent, a soothing agent and a tonicity agent, which can be formulated according to a common procedure by blending materials generally used for formulating a medicinal formulation. Examples of such non-toxic materials available include, for example, animal and vegetable oils such as soybean oil, beef tallow and synthetic glyceride; for example, hydrocarbons such as liquid paraffin, squalane and solid paraffin; for example, ester oils such as octyldodecyl myristate and isopropyl myristate; for example, higher alcohols such as cetostearyl alcohol and behenyl alcohol; silicon resin; silicon oil; for example, surfactants such as polyoxyethylene fatty acid ester, sorbitan fatty acid ester, glycerine fatty acid ester, polyoxyethylene sorbitan fatty acid ester, polyoxyethylene hardened caster oil and polyoxyethylene-polyoxypropylene block copolymer; for example, water-soluble polymers such as hydroxyethylcellulose, polyacrylic acid, carboxy vinyl polymer, polyethylene glycol, polyvinyl pyrrolidone and methylcellulose; for example, lower alcohols such as ethanol and isopropanol; for example, polyol such as glycerine, propylene glycol, dipropylene glycol, sorbitol and polyethylene glycol; for example, saccharides such as glucose and sucrose; for example, inorganic powers such as anhydrous silicon, magnesium aluminum silicate and aluminum silicate; inorganic salts such as sodium chloride and sodium phosphate; and purified water.

Examples of excipients include, for example, lactose, fructose, cornstarch, white sugar, glucose, mannitol, sorbit, crystalline cellulose and silicon dioxide; examples of binders include, for example, polyvinyl alcohol, polyvinyl ether, methylcellulose, ethylcellulose, gum arabic, tragacanth, gelatin, shellac, hydroxypropylmethylcellulose, hydroxypropylcellulose, polyvinyl pyrrolidone, polypropyleneglycol polyoxyethylene block copolymer and meglumine; examples of disintegrating agents include, for example, starch, agar, gelatin powder, crystalline cellulose, calcium carbonate, sodium hydrogen carbonate, calcium citrate, dextrin, pectin and calcium carboxymethylcellulose; examples of lubricants include, for example, magnesium stearate, talc, polyethylene glycol, silica and hardened plant oil; examples of colorants include pharmaceutically acceptable additives; and examples of flavoring or aromatic substances include cocoa powder, menthol, aromatic powder, mint oil, borneol and cinnamon powder. The materials mentioned above may be salts or solvates thereof.

An oral formulation is produced, for example, into powder, fine granule, granule, a tablet, a coated tablet, a capsule or the like obtained according to a routine procedure after adding an excipient, and if necessary, further a binder, a disintegrating agent, a lubricant, a colorant, a flavoring or aromatic substance or the like to a compound having a ChE inhibitory activity, a salt or a solvate thereof, or a prodrug thereof, a salt or a solvate thereof.

Tablets and granules may be coated according to a well-known method using a coating agent such as carnauba wax, hydroxypropylmethylcellulose, macrogol, hydroxypropylmethyl phthalate, cellulose acetate phthalate, white sugar, titanium oxide, sorbitan fatty acid ester or calcium phosphate.

Specific examples of carrier used for producing a syrup agent include sweetening agents such as white sugar, glucose and fructose, suspending agents such as gum arabic, tragacanth, carmellose sodium, methylcellulose, sodium alginate, crystalline cellulose and veegum, and dispersants such as sorbitan fatty acid ester, sodium lauryl sulphate and polysorbate 80. For production of syrup, a flavoring material, an aromatic material, a preservative, a solubilizing agent and a stabilizer can be added as may be necessary. The product may be in a form of dry syrup that can be dissolved or suspended upon use.

An injectable agent is generally prepared by dissolving, for example, a salt of a compound having a ChE inhibitory activity in injectable distilled water, and may be formulated according to a common procedure by adding a solubilizing agent, a buffer, a pH regulator, a tonicity agent, a soothing agent, an antiseptic, a preservative, a stabilizer or the like as may be necessary.

The injectable agent may be asepticized by filter sterilization using a filter or by addition of a disinfectant. The injectable agent may be produced into a form that can be prepared upon use. Specifically, the injectable agent may be prepared into a sterile solid composition by lyophilization or the like which can be dissolved in sterile injectable distilled water or other solvent before use.

Production of an external medicine is not limited to a particular production procedure and may be produced by any routine procedure. Various materials generally used in pharmaceuticals, medicated cosmetics, cosmetics or the like may be used as a base material. For example, materials such as animal or plant oil, mineral oil, ester oil, wax, higher alcohols, fatty acids, silicon oil, surfactant, phospholipids, alcohols, polyols, water-soluble polymers, clay minerals, purified water or the like, and if necessary, a pH regulator, an antioxidant, a chelating agent, an antiseptic, a fungicide, a colorant, an aromatic substance or the like may also be added. As to an inhaler, a compound having a ChE inhibitory activity, a salt or a solvent thereof, or a prodrug thereof or a salt or a solvent thereof can be delivered with an injector, a nebulizer, a pressurized package or other means suitable for delivering aerosol spray for inhalation administration. The pressurized package may contain an appropriate propellant. Moreover, for inhalation administration, a compound having a ChE inhibitory activity, a salt or a solvate thereof, or a prodrug thereof, a salt or a solvate thereof may be administered in a form of dry powdered composition or liquid spray. For administration with an adhesive patch via transdermal absorption, it is preferable to select a so-called free-form that does not form salt. For topical application to skin, a compound having a ChE inhibitory activity may be formulated into ointment, cream or lotion or as an active ingredient in a transdermal patch. Ointment and cream can be formulated, for example, by adding an appropriate thickening agent and/or gelling agent to an aqueous or oil base. Lotion can be formulated by using an aqueous or oil base and may generally contain one or more of an emulsifying agent, a stabilizer, a dispersant, a suspending agent, a thickening agent and/or a colorant. The compound having a ChE inhibitory activity may also be administered by ion transfer therapy.

If necessary, components such as a blood circulating agent, a disinfectant, an anti-inflammatory agent, a cellular stimulant, vitamins, amino acids, a moisturizing agent, a keratolytic agent may further be blended. The proportion of the active ingredient to the carrier varies between 1 to 90% by weight.

The overactive bladder therapeutic agent used in the method of the present invention can generally include, as an active ingredient, a compound having a ChE inhibitory activity, a salt or a solvate thereof, or a prodrug thereof, a salt or a solvate thereof at a proportion of 0.5% by weight or more, preferably 10 to 70% by weight.

When the compound having a ChE inhibitory activity, a salt or a solvate thereof, or a prodrug thereof, a salt or a solvate thereof is used for the treatment described above, it is purified for at least 90% or more, preferably 95% or more, more preferably 98% or more, still more preferably 99% or more.

A dose of the compound having a ChE inhibitory activity, a salt or a solvate thereof, or a prodrug thereof, a salt or a solvate thereof for oral administration varies as it is determined according to multiple factors including, for example, administration route, type of disease, degree of symptom, patient's age, sex and weight, type of salt, specific type of disease, pharmacological aspects such as pharmacokinetics and toxicological features, use of drug delivery system, and whether it is administered concomitantly with other drugs, but one skilled in the art will be able to determine appropriately. For example, for an adult (60 kg), about 0.001 to 1000 mg/day, preferably about 0.01 to 500 mg/day, and more preferably about 0.1 to 300 mg/day can be administered at one time or in several times. When administered to a child, a dose is possibly lower than that for an adult. The administration procedure actually used may widely vary and can depart from the preferable administration procedures described herein. For example, in the case of donepezil hydrochloride, preferably about 0.1 to 300 mg/day, more preferably about 0.1 to 100 mg/day, and still more preferably about 1.0 to 50 mg/day can be administered to an adult (weight 60 kg). In a preferred embodiment of donepezil hydrochloride, a 5 mg or 10 mg donepezil hydrochloride tablet commercially available under the trade name of Aricept tablet (Eisai Co., Ltd.), or donepezil hydrochloride under the trade name of Aricept fine granule (Eisai Co., Ltd.) can be administered. For example, tablets may be administered 1 to about 4 times a day. In a preferred embodiment, a 5 mg or 10 mg Aricept tablet (Eisai Co., Ltd.) is administered once a day. Those skilled in the art will appreciate that when donepezil hydrochloride is administered to a child, the dose thereof is possibly lower than that for an adult. In a preferred embodiment, donepezil hydrochloride can be administered to a child for about 0.5 to 10 mg/day, preferably about 1.0 to 3 mg/day. Preferably, in the case of Tacrine, about 0.1 to 300 mg/day, preferably about 40 to 120 mg/day is administered to an adult (weight 60 kg); in the case of Rivastigmine, about 0.1 to 300 mg/day, preferably about 3 to 12 mg/day is administered to an adult (weight 60 kg); in the case of galantamine, about 0.1 to 300 mg/day, preferably about 16 to 32 mg/day is administered to an adult (weight 60 kg); and in the case of physostigmine, about 0.1 to 300 mg/day, preferably about 0.6 to 24 mg/day is administered to an adult (weight 60 kg). For each of the above cases, a dose to a child may possibly be lower than that for an adult.

As to parenteral administration, a preferable dose for adhesive patch would be about 5 to 50 mg/day, more preferably about 10 to 20 mg/day for an adult (60 kg). An injectable agent may be produced by dissolving or suspending it in a pharmacologically acceptable carrier such as saline or a commercially available injectable distilled water to a concentration of 0.1 μg/ml carrier to 10 mg/ml carrier. A dose of such an injectable agent to a patient in need of the treatment may be about 0.01 to 50 mg/day, preferably about 0.01 to 5.0 mg/day, more preferably about 0.1 to 1.0 mg/day for an adult (60 kg), and may be administered 1 to 3 times a day. When administered to a child, the dose may possibly be lower than that for an adult.

3. Process for Screening Substance for Suppressing Overactive Bladder Resulting from Cerebral Infarction, Pharmacologically Acceptable Salt or Solvate Thereof.

The present invention further provides a process for screening a substance that suppresses overactive bladder resulting from cerebral infarction, a pharmacologically acceptable salt or a solvate thereof.

A screening process according to the present invention comprises administrating a candidate substance to a non-human mammal, and detecting or determining a change in a phenotype of overactive bladder resulting from cerebral infarction in the presence and absence of the candidate substance.

Herein, “in the presence” means that the candidate substance has been administered to a non-human animal, and “in the absence” means that the candidate substance has not been administered to a non-human animal. Thus, upon screening, individuals from a non-human animal group administered with the candidate substance are compared with individuals from a control non-human animal group not administered with the candidate substance to detect or determine the phenotypes. Alternatively, a phenotype of an individual prior to administration of a candidate substance may be compared with a phenotype of the same individual administered with the candidate substance.

According to the screening process of the present invention, the candidate substance include a substance having an ChE (including ACHE) inhibitory activity, for example, the compound having a ChE inhibitory activity described above, an anti-ChE antibody, siRNA and shRNA to ChE and the like. The substance may be a salt or a solvate of the above. The compound having a ChE inhibitory activity can be produced or obtained by referring to the description above. The anti-ChE antibody may be either a monoclonal antibody or a polyclonal antibody, and those skilled in the art would be able to produce such antibodies, for example, by using ChE as a sensitized antigen. siRNA or shRNA for ChE gene may be any nucleic acid that is capable of suppressing the expression of ChE gene, and those skilled in the art would be able to appropriately design a sequence and produce siRNA or shRNA (Elbashir, S. M., et. al., Genes Dev., 15, 188-200, 2001).

A candidate compound may be administered to a non-human mammal either orally or parenterally.

A change in a phenotype of overactive bladder resulting from cerebral infarction may use at least one of a bladder capacity, a bladder contraction pressure and an amount of retained urine as an index. The substance can be determined to be suppressive to overactive bladder resulting from cerebral infarction when at least one of the following (a) to (c) applies:

(a) when the substance increases bladder capacity,

(b) when the substance prevents bladder contraction pressure from decreasing, or

(c) when the substance prevents retained urine from increasing.

In order to detect or determine the change in a phenotype of the overactive bladder resulting from cerebral infarction, a pressure within the bladder is determined, preferably a pressure within the non-human mammal bladder in a waking state is determined.

The present invention further provides a kit for screening a substance capable of suppressing overactive bladder resulting from cerebral infarction, a pharmacologically acceptable salt or a solvate thereof which are to be used in the method described above. The screening kit of the invention includes means required for determining a change in a phenotype of overactive bladder resulting from cerebral infarction. Agents suitably used upon determining the phenotype change are general anesthetics (e.g., halothane) and saline. The screening kit of the present invention may further include an instruction, a tube, a flask or the like.

### EXAMPLES

Hereinafter, the present invention will be described more specifically by way of non-limiting examples.

### Example 1

### Production of Donepezil Hydrochloride

### (a) Synthesis of 1-benzyl-4-piperidine carboaldehyde

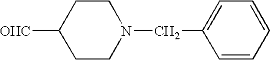

26.0 g of methoxymethylenetriphenylphosphonium chloride was suspended in 200 ml anhydrous ether, and 1.6 M n-butyllithiumhexane solution was added dropwise at room temperature. After stirring at room temperature for 30 minutes, the resultant was cooled to 0° C., and 14.35 g 1-benzyl-4-piperidone in 30 ml anhydrous ether solution was added. After stirring at room temperature for 3 hours, insoluble matter was filtered out and the filtrate was concentrated under reduced pressure. The obtained residue was dissolved in ether and extracted with 1N hydrochloric acid. Following adjustment of pH to 12 with sodium hydroxide solution, the resultant was extracted with methylene chloride. The resultant was dried with magnesium sulfate, and concentrated under reduced pressure. The obtained residue was purified through a silica gel column, thereby obtaining 5.50 g of oily substance (yield 33%).

Subsequently, the obtained oily substance was dissolved in 40 ml methanol, and added with 40 ml 1N hydrochloric acid. The reaction solution was heated to reflux for 3 hours, then concentrated under reduced pressure. The residue was dissolved in water. Thereafter, pH of the dissolved solution was adjusted to 12 with sodium hydroxide solution, and extracted with methylene chloride. The extracted solution was washed with saturated saline, dried with magnesium sulfate, and concentrated under reduced pressure. The obtained residue was purified through a silica gel column to obtain 2.77 g of 1-benzyl-4-piperidinecarboaldehyde (yield 54%) as an oily substance.

The structure of the obtained compound was determined by NMR.

Molecular formula; C₁₃H₁₇NO ¹H-NMR (CDC l₃)δ; 1.40-2.40 (7H, m), 2.78 (2H, dt), 3.45 (2H, s), 7.20 (5H, s), 9.51 (1H, d).

### (b) Synthesis of 1-benzyl-4-[(5,6-dimethoxy-1-indanone)-2-ylidenyl]methylpiperidine.hydrochloride (formula below)

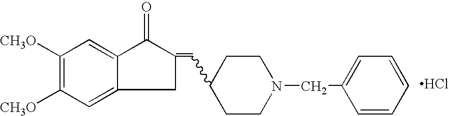

This reaction took place in argon atmosphere.

2.05 ml diisopropylamine was added to 10 ml anhydrous THF and 9.12 ml 1.6M n-butyllithium hexane solution was further added at 0° C. The resultant was stirred at 0° C. for 10 minutes, cooled to −78° C., and added with 2.55 g 5,6-dimethoxy-1-indanone in 30 ml anhydrous THF solution and 2.31 ml hexamethyl phosphoramide. The resultant was stirred at −78° C. for 15 minutes, added with 2.70 g 1-benzyl-4-piperidinecarboaldehyde obtained in (a) in 30 ml anhydrous THF solution, and gradually heated to room temperature. Again stirring at room temperature for another 2 hours, 1% ammonium chloride solution was added to separate the organic layer. Next, the aqueous layer was extracted with ethyl acetate, combined with the organic layer separated above, and washed with saturated saline. The solution was dried with magnesium sulfate, concentrated under reduced pressure, and the obtained residue was purified through a silica gel column (methylene chloride:methanol=500:1 to 100:1). After concentrating the eluate under reduced pressure, the resultant was dissolved in methylene chloride, added with 10% hydrochloric acid-ethyl acetate solution, and further concentrated under reduced pressure to obtain a crystal. This was recrystallized from methylene chloride-IPE to obtain 3.40 g of 1-benzyl-4-[(5,6-dimethoxy-1-indanone)-2-ylidenyl]methylpiperidine hydrochloride (yield 62%) having the following properties:

Melting point (° C.); 237-238 (dec.)

Elementary analysis; as C₂₄H₂₇NO₃ HCl, CHN calculated (%): 69.64 6.82 3.38, found (%): 69.51 6.78 3.30.

### (c) 1-benzyl-4-[(5,6-dimethoxy-1-indanone)-2-yl]methylpiperidine hydrochloride

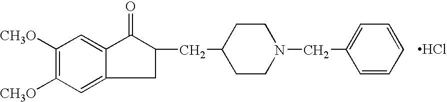

0.40 g of 1-benzyl-4-[(5,6-dimethoxy-1-indanone)-2-ylidenyl]methylpiperidine obtained in (b) was dissolved in 16 ml THF, and 0.04 g of 10% palladium-carbon was added. After hydrogenating under atmospheric pressure at room temperature for 6 hours, catalyst was filtered out, and the filtrate was concentrated under reduced pressure. The residue was purified through a silica gel column (methylene chloride:methanol=50:1), and the eluate was concentrated under reduced pressure. Thereafter, the residue was dissolved in methylene chloride, added with 10% hydrochloric acid-ethyl acetate solution, and was further concentrated under reduced pressure, thereby obtaining a crystal. This was recrystallized from ethanol-IPE to obtain 0.36 g of 1-benzyl-4-[(5,6-dimethoxy-1-indanone)-2-yl]methylpiperidine hydrochloride (donepezil hydrochloride) (yield 82%) having the following properties:

Melting point (° C.); 211-212 (dec.)

Elementary analysis; as C₂₄H₂₉NO₃ HCl, CHN calculated (%): 69.30 7.27 3.37, found (%): 69.33 7.15 3.22.

### Example 2

### Cerebral Infarction Model

10-week-old female SD rats (purchased from Sankyo Labo Service Co.) were subjected to cystostomy under anesthesia of 1.5% halothane. They were further subjected to cervical incision, and a nylon suture was inserted into their left internal carotid to produce cerebral infarction by middle cerebral artery embolization. These cerebral infarction rats were restrained in Bollman cages in waking state. Measurement of the pressures within their bladders indicated significant decrease in the bladder capacities (Yokoyama O, Yoshiyama M, Namiki M, de Groat W C: Influence of anesthesia on bladder hyperactivity induced by middle cerebral artery occlusion in the rat. Am J Physiol 273: R1900-R1907, 1997). On the other hand, decrease in bladder capacity was not seen in sham-operation rats which were just subjected to common carotid decortication. In the examples below, experiments were conducted using overactive bladder model of this cerebral infarction model.

### Experiments Using Cerebral Infarction Models: Cholineacetyltransferase Activity

The rats were sacrificed 1, 5 and 24 hours after producing the left middle cerebral artery embolization as described above. Right and left cortices, right and left hippocampi and pons were separated immediately to determine cholineacetyltransferase activity at each site.

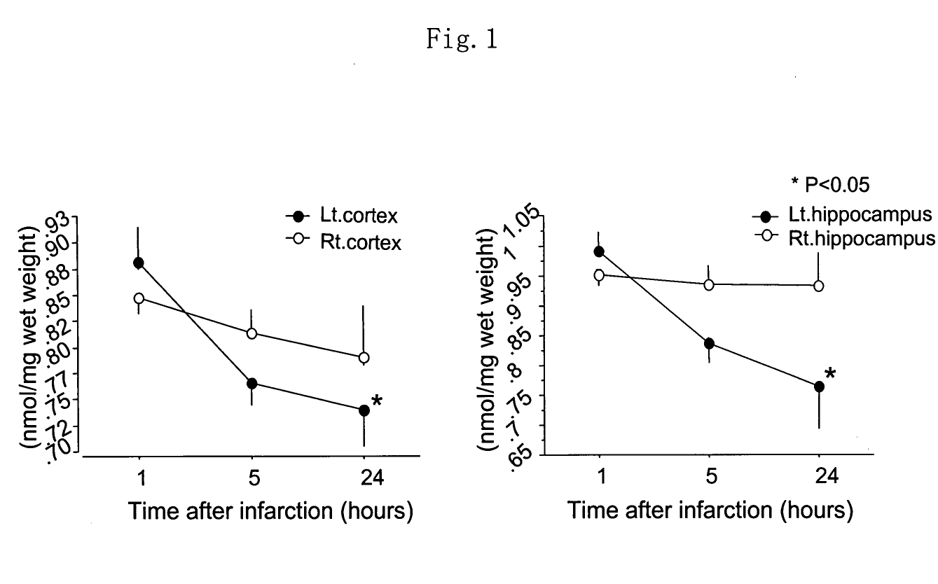

Results are shown in FIG. 1. The left panel in FIG. 1 shows the results of left (Lt) and right (Rt) cortices, while the right panel in FIG. 1 shows the results of left (Lt) and right (Rt) hippocampi. The vertical axis represents the cholineacetyltransferase activity (nmol/mg wet weight) while the horizontal axis represents time (hour) after infarction. As a result, cholineacetyltransferase activity was decreased 24 hours after the cerebral infarction on the infarction side, i.e., in the left cortex (Lt. cortex) and the left hippocampus (Lt. hippocampus) (FIG. 1).

### Example 3

### Experiments Using Cerebral Infarction Models: Intravenous Administration of Donepezil Hydrochloride

Donepezil hydrochloride (ARICEPT® supplied from Eisai Co., Ltd.) was dissolved in saline, which was intravenously administered to overactive bladder model rats at 5×10⁻⁷ mg/kg to 5×10⁻¹ mg/kg every 30 minutes. Bladder capacities of rats administered with various concentrations of donepezil hydrochloride were determined.

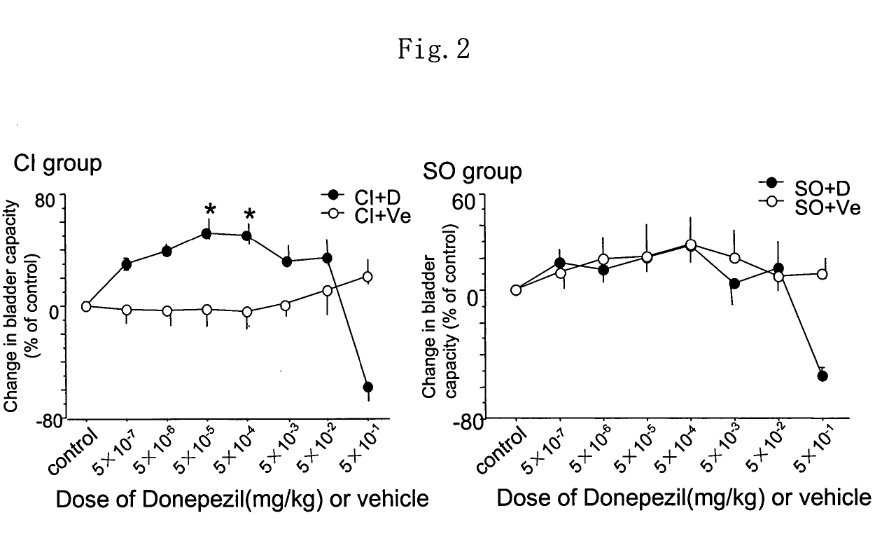

Results are shown in FIG. 2. In FIG. 2, changes in the bladder capacities upon administration of donepezil hydrochloride (“+D”) or vehicle (“+Ve”) at respective concentrations are shown as percentages of change from that of a control. The left panel shows the results for cerebral infarction model rats (CI group) while the right panel shows the results for sham-operation rats (SO group). As a result, transvenous administration of donepezil hydrochloride to the cerebral infarction model rats (CI group) increased the bladder capacities (FIG. 2). Donepezil hydrochloride doses of 5×10⁻⁴ mg/kg and 5×10⁻⁵ mg/kg showed significant difference from the vehicle administration group. In particular, the dose of 5×10⁻⁵ mg/kg showed an increase of 52.8% (p=0.0163). On the other hand, increase of 27.3% was observed in the sham-operation rats (SO group) upon donepezil hydrochloride administration, but difference from the vehicle administration group was not significant. In addition, no change in the micturition contraction pressure and little retained urine were observed at the donepezil hydrochloride doses of 5×10⁻⁴ mg/kg and 5×10⁻⁵ mg/kg.

### Example 4

### Experiments Using Cerebral Infarction Models: Intraventricular Administration of Donepezil Hydrochloride

After producing cerebral infarction rats, drug infusion tubes were placed in the lateral ventricles of the rats. After the rats came out from under anesthesia, 5 μl of donepezil hydrochloride at concentrations of 5×10⁻⁷ mg/ml to 5×10⁻⁴ mg/ml were intraventricularly administered to them to examine the effects on the bladder functions.

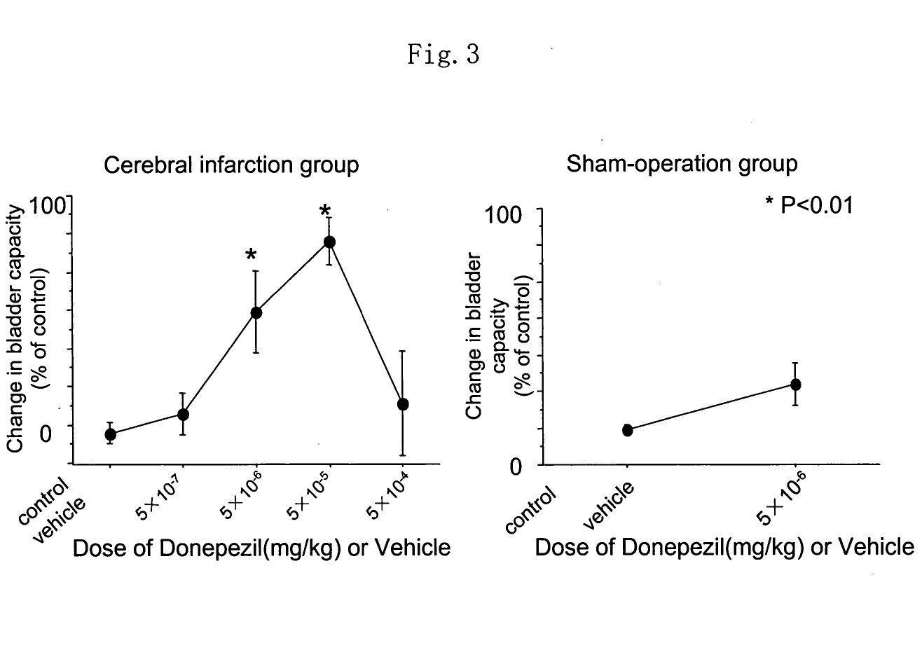

Results are shown in FIG. 3. As a result, bladder capacities increased upon intraventricular administration of donepezil hydrochloride in both cerebral infarction rats and sham-operation rats. Specifically, when 5 μl of donepezil hydrochloride at 5×10⁻⁵ mg/ml and 5×10⁻⁶ mg/ml were administered to the cerebral infarction rats, bladder capacity increased with a significant margin from the vehicle administration group. At an administration of 5×10⁻⁵ mg/ml, the increase was 95.8% (p=0.0088). No change was seen in the micturition contraction pressure.

### Example 5

In this example, a cholinesterase inhibitor is administered to rats with overactive bladder resulting from cerebral infarction to examine change in their bladder functions.

Recently, ischemia caused in the brain is reported to be associated with deterioration of functions of acetylcholine system in the brain. Therefore, activation of ACh system in the brain is suggested to recover higher brain functions and further ameliorate overactive bladder such as bladder irritation.

Thus, in this example, cerebral infarction rats are experimentally produced and donepezil hydrochloride, i.e., a central cholinesterase inhibitor used as a treatment drug for dementia of the Alzheimer type, is administered to them at different doses so as to examine its effects on the bladder functions.

Materials and Methods

10-week-old female SD rats were subjected to cystostomy under anesthesia of 1.5% halothane. After waking, they were restrained in Bollman cages and pressures within their bladders were measured. Then, again under halothane anesthesia, a 4-0 nylon suture was inserted into left middle cerebral artery through common carotid to produce cerebral infarction rats (these rats are referred to as “CI”). On the other hand, those with only decortication of common carotid were produced as a sham-operation group (these rats are referred to as “SO”).

Donepezil hydrochloride was dissolved in saline and intravenously administered at 5×10⁻⁷ mg/kg to 5×10⁻¹⁻mg/kg every 30 minutes from an hour after the operation. Similarly, a group of rats subjected to cerebral infarction and sham-operation, and administered with saline was produced as well, as a vehicle.

Donepezil hydrochloride is indicated as D while vehicle administered with saline is indicated as Ve. In the four groups CI+D, CI+Ve, SO+D and SO+Ve, 5 examples per group, i.e., total of 20 examples were produced to determine change in their bladder capacities and micturation contraction pressures. Mann-Whitney's U test was employed.

In addition, functions of ACh system in each site of the brain after cerebral infarction were assessed. Cerebral infarction was produced in 10-week-old female SD rats, which were sacrificed 1, 5 and 24 hours after the infarction to determine cholineacetyltransferase activities in each site of the brain, the right and left cortices, the right and left hippocampi and the stem area.

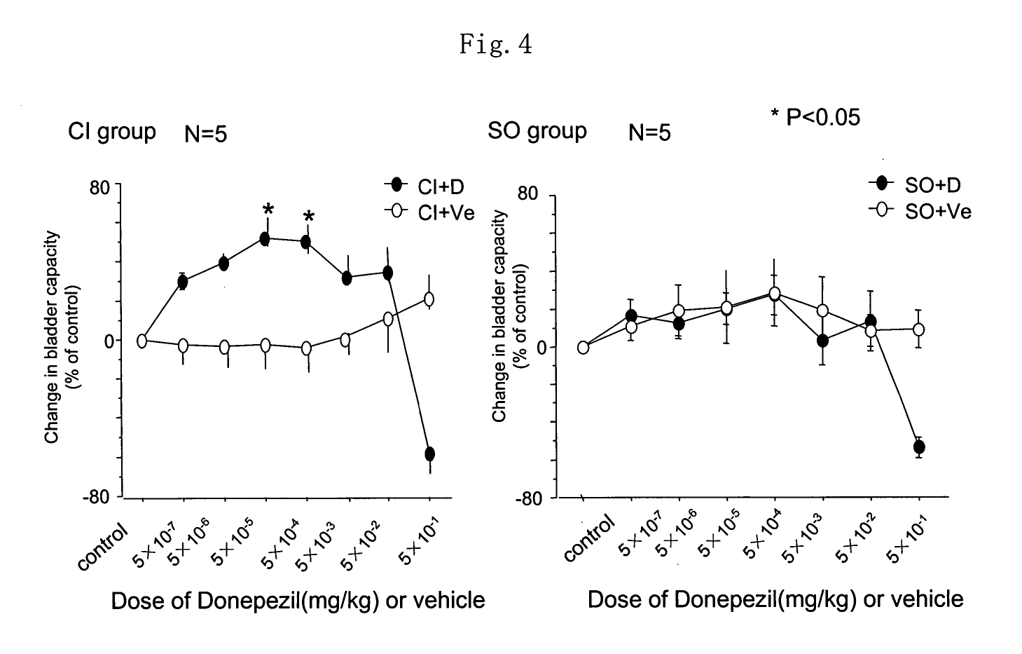

Results are shown in FIG. 4. FIG. 4 shows change in the bladder capacity after the cerebral infarction. In the CI group, bladder capacity gradually increased after the administration of donepezil hydrochloride, and the bladder capacity most increased at 5×10⁻⁴ to 5×10⁻⁵ mg/kg. The percentage of change was 52.8%, and difference from vehicle was significant.

In the SO group, bladder capacity increased by donepezil administration but gradually decreased after administration at 5×10⁻⁴ mg/kg.

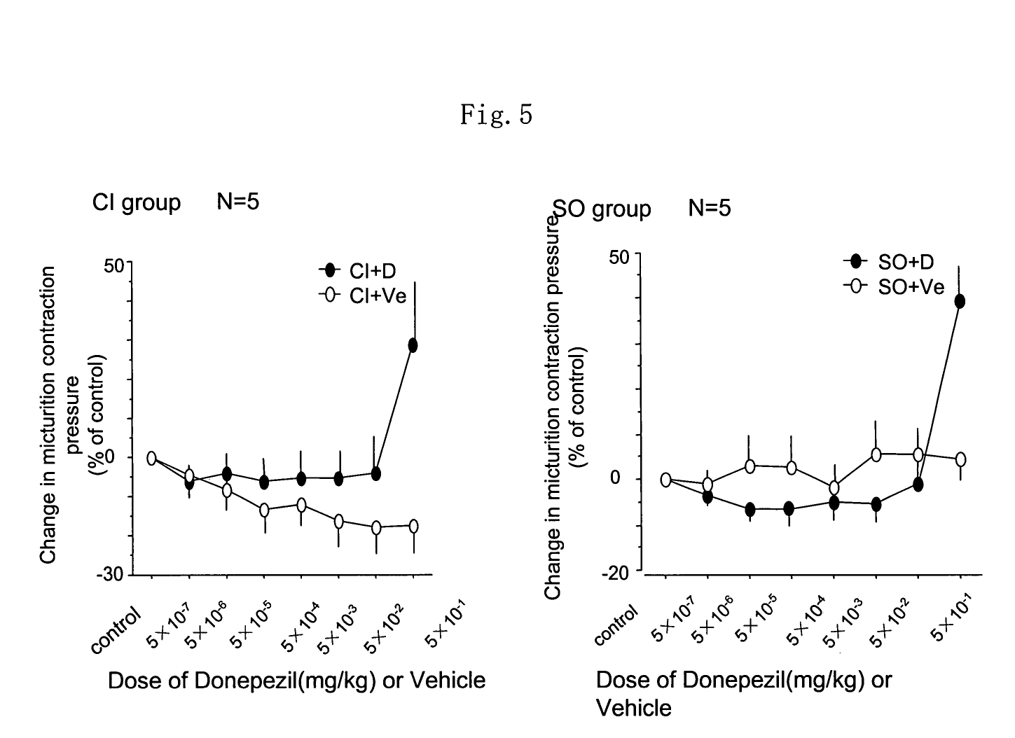

The change in the micturition contraction pressure is shown in FIG. 5. Referring to FIG. 5, in both of the CI group and the SO group, no significant difference is seen between the donepezil hydrochloride administration group and the vehicle administration group at 5×10⁻⁷ to 5×10⁻² mg/kg and the contraction pressure significantly increased at 5×10⁻¹ mg/kg in the group administered with donepezil.

Subsequently, difference of cerebral infarction areas between the donepezil administration group and the saline administration group in the CI group were compared. Brains were removed after 6 hours following cerebral infarction. Thereafter, brains were cut out into coronal sections at 2 mm intervals, which were stained with 2% TTC (2,3,5-triphenyl-tetrazolium chloride) solution to determine the cerebral infarction areas.

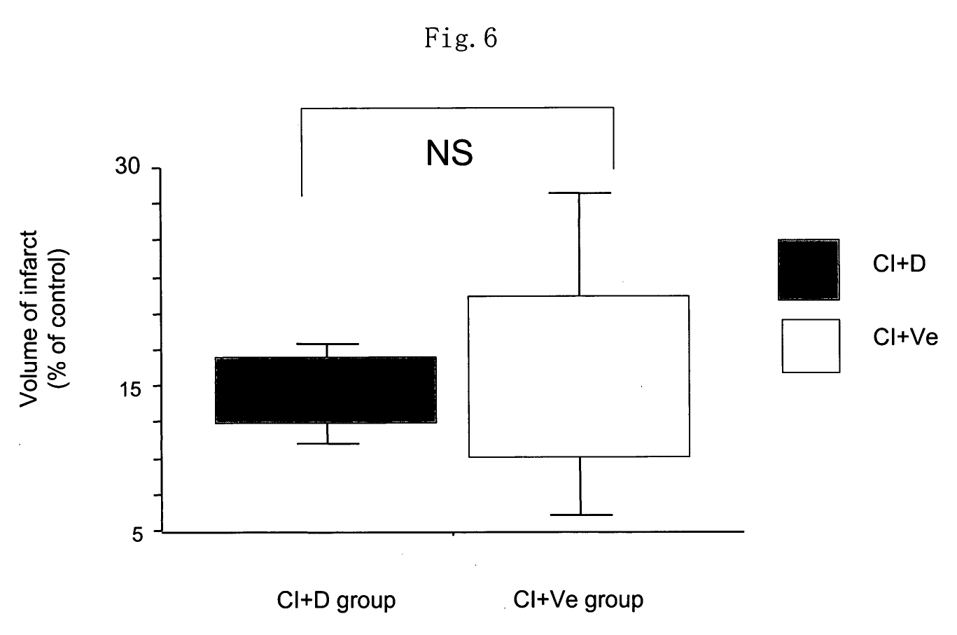

Results are shown in FIG. 6. FIG. 6 shows percentage of a volume of cerebral infarct to that of the entire brain. No significant difference was seen between the two groups.

Subsequently, cholineacetyltransferase activities in the right and left cortices and right and left hippocampi were compared at 1, 5 and 24 hours after cerebral infarction.

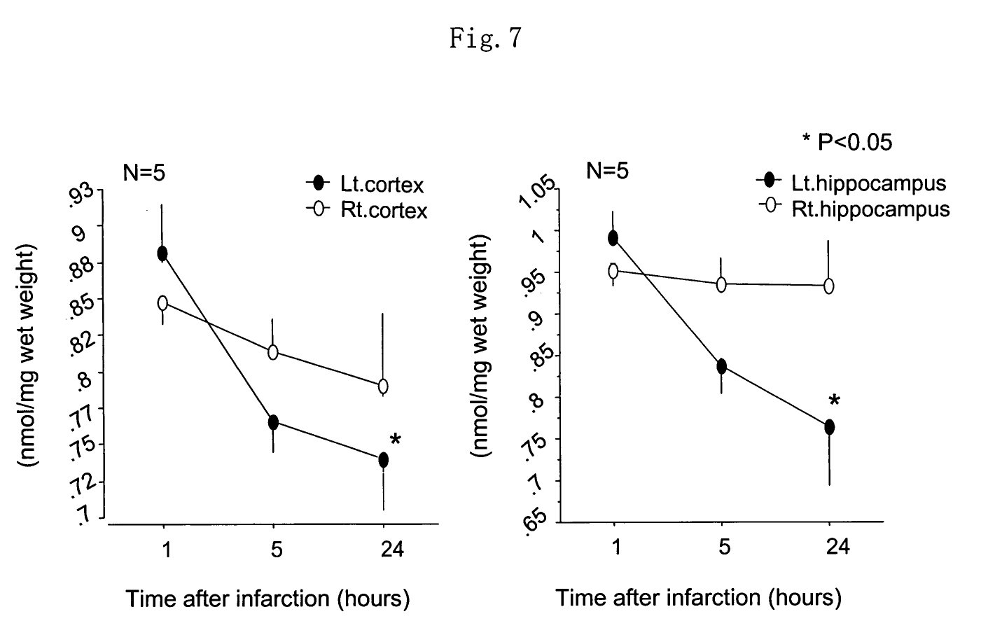

Results are shown in FIG. 7. As can be appreciated from FIG. 7, cholineacetyltransferase activity was significantly decreased in the right and left cortices (left panel) as well as in the left hippocampi (right panel) after 24 hours than after 1 hour.

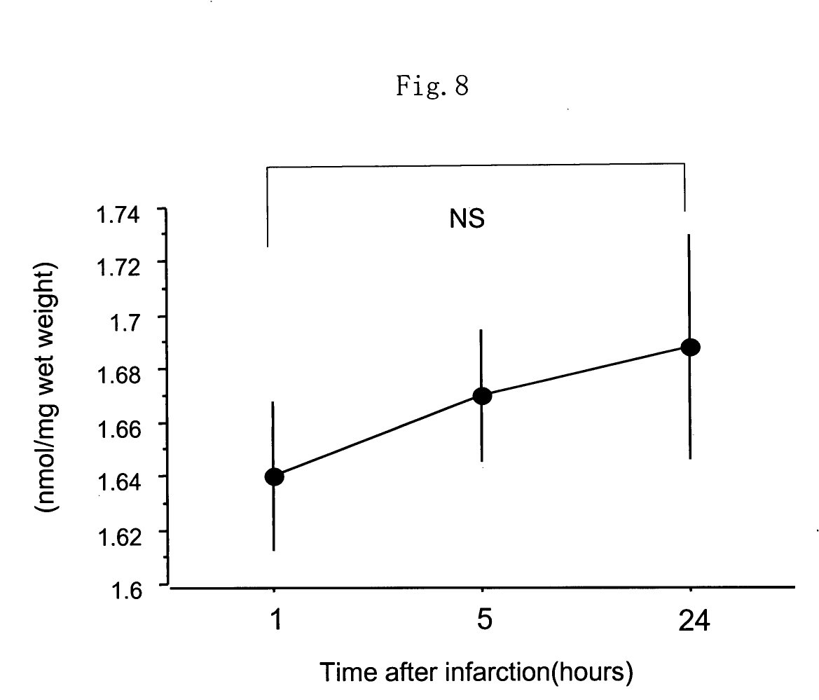

The cholineacetyltransferase activity was also examined in the pons but no significant difference was seen between 1 hour and 24 hours (FIG. 8).

### Example 6

Example 5 showed that donepezil hydrochloride ameliorated overactive bladder caused after cerebral infarction and that acetylcholine in the brain was decreased by cerebral infarction. Therefore, in this example, donepezil hydrochloride was intraventriculary administered to confirm whether the overactive bladder was actually ameliorated with donepezil hydrochloride that activates acetylcholine decrease caused by cerebral infarction.

Cerebral infarction rats were produced in the same manner as Example 5 to examine the effect of intraventricularly administered donepezil hydrochloride on the bladder functions.

The process was conducted in the same manner as Example 5 except that metal tubes were inserted into lateral ventricles upon producing cerebral infarction rats. Pressure within the bladder was measured at the moment of waking, and 2 hours later donepezil hydrochloride was intraventricularly administered via the tubes placed. Feasibility of intraventricular administration was determined after injecting ink into the brain, sacrificing the rats and removing the brains to verify on their faces. Concentrations of donepezil hydrochloride were 5×10⁻⁷ mg/ml to 5×10⁻⁴ mg/ml. As vehicle, saline was infused for 5 μml. Experiments of five examples for each concentration were conducted.

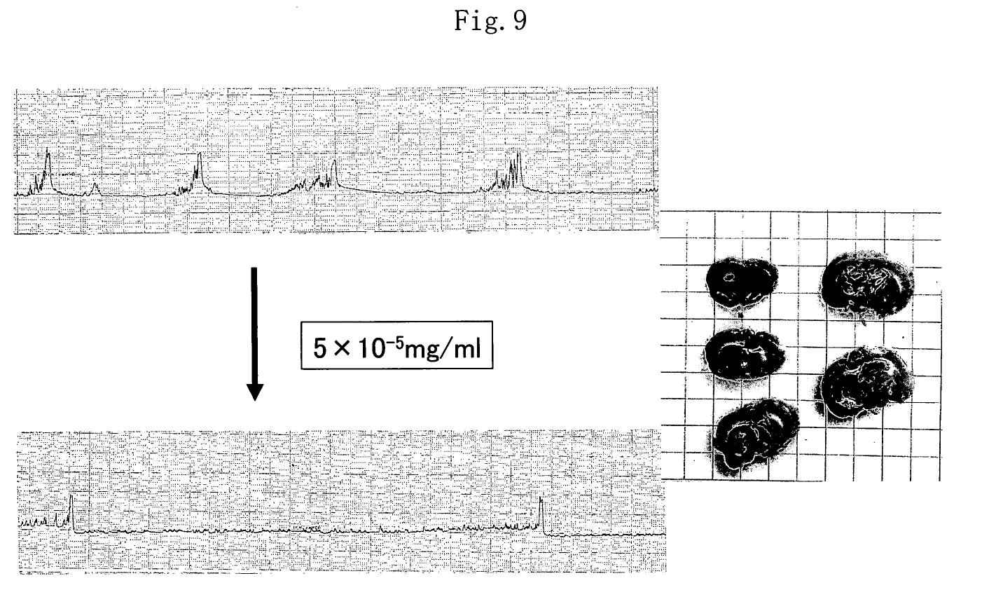

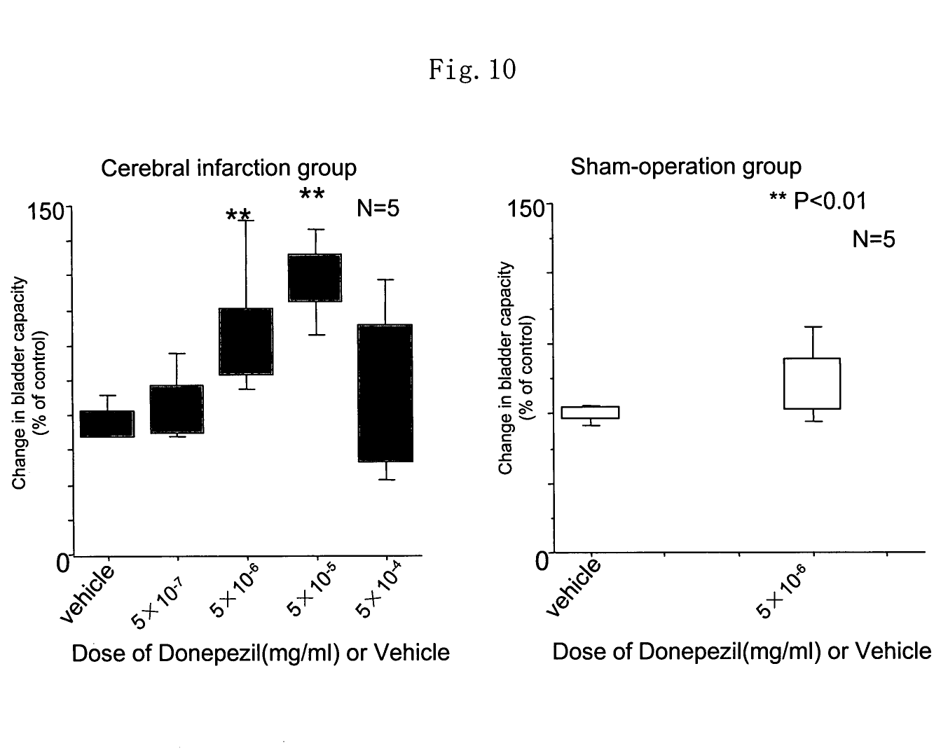

Results are shown in FIGS. 9 and 10. FIG. 9 shows one exemplary curve of pressure within the bladder intraventricularly administered with donepezil hydrochloride (concentration for administration: 5×10⁻⁵ mg/ml). As shown in FIG. 10, the bladder capacity notably increased after the administration of donepezil hydrochloride. The right panel in FIG. 9 shows brains removed after sacrifice. The site of cerebral infarction was not stained with TCC solution while ink was infused in the ventricle.

As to the pressure within the bladder, the bladder capacity was most increased at an administration of 5×10⁻⁵ mg/ml in the cerebral infarction group, and difference from the vehicle group was significant at 5×10⁻⁵ mg/ml and 5×10⁻⁶ mg/ml (p=0.0088) (FIG. 10, left panel). In the sham-operation group, comparison with the vehicle group at a concentration of 5×10⁻⁵ mg/ml that most increased the bladder capacity showed a tendency for the bladder capacity to increase in the donepezil group (p=0.93) (FIG. 10, right panel).

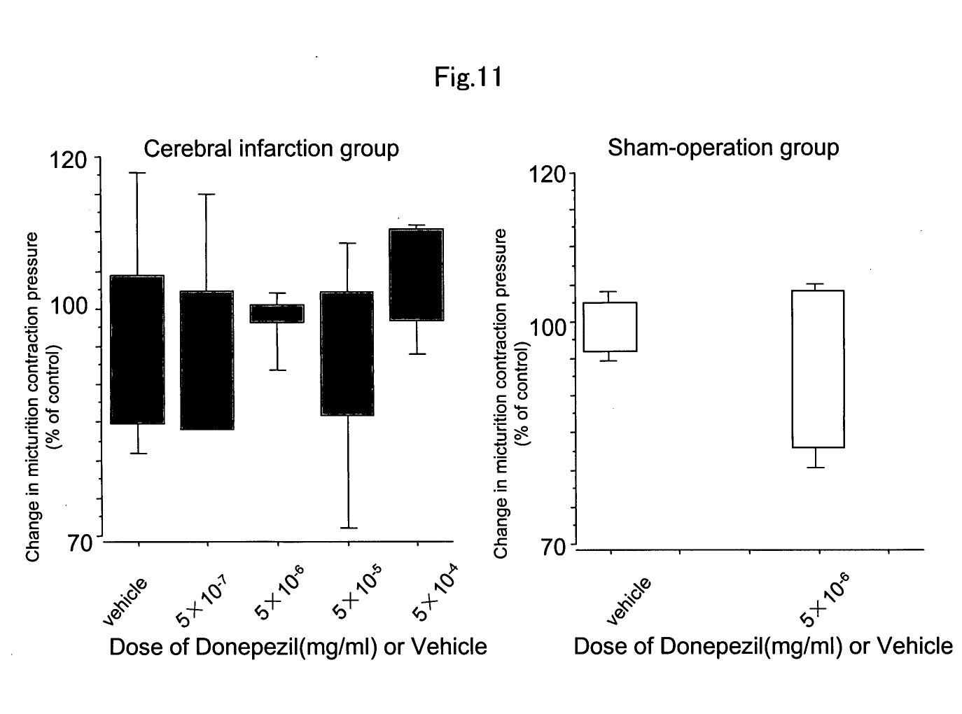

As to the change in the micturition contraction pressure, no significant difference was seen between donepezil hydrochloride and vehicle at respective doses (FIG. 11).

### Example 7

In Example 6, increase in the bladder capacity was also seen for intraventricular administration confirming again that donepezil hydrochloride is selective to brain. In this example, in order to confirm that donepezil hydrochloride acts superior to pons, brain was removed after cerebral infarction and donepezil hydrochloride was administered.

Similar to Examples 5 and 6, following cystostomy, rats were classified into cerebral infarction group and sham-operation group, pressures within the bladders were measured, brains were removed and 5×10⁻⁵ mg/ml of donepezil hydrochloride or vehicle was transvenously administered.

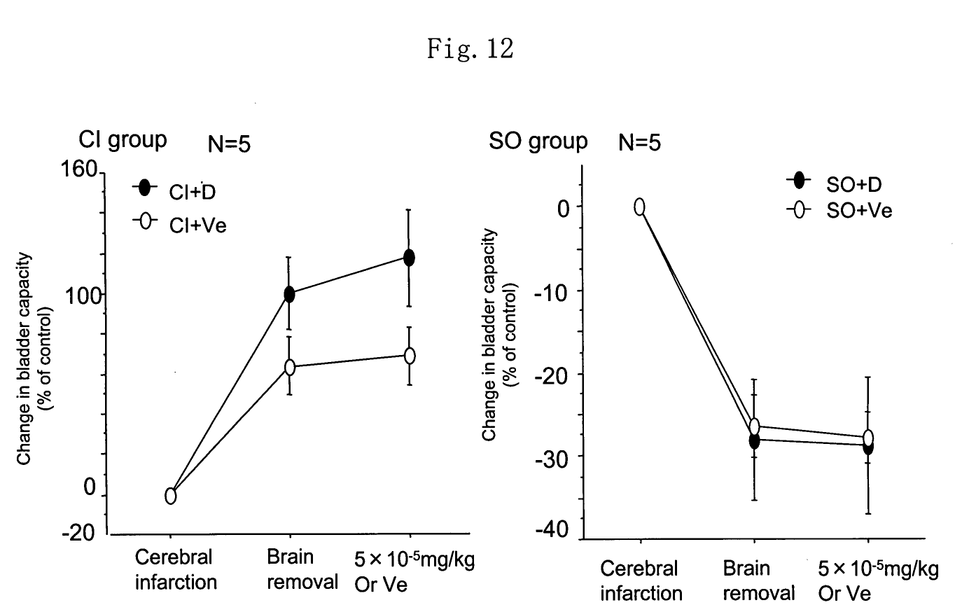

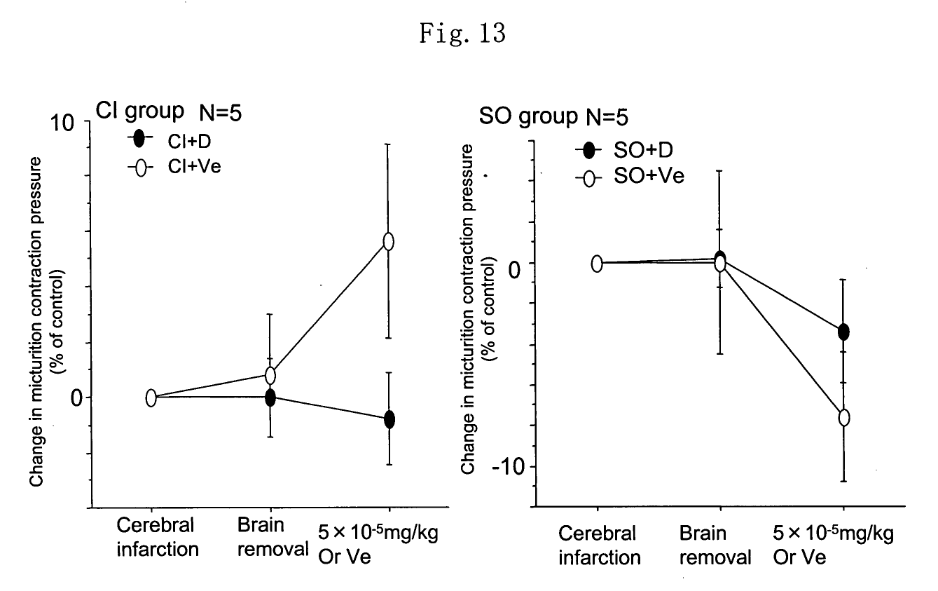

Results are shown in FIGS. 12 and 13.

As a result of the pressure measurements within the bladders, bladder capacity increased after removal of the brains from rats in the cerebral infarction group, and no increase was observed upon the subsequent donepezil hydrochloride administration (FIG. 12, left panel). In the sham-operation group, bladder capacity decreased upon brain removal but did not change with donepezil hydrochloride (FIG. 12, right panel). Retained urine was not observed in any of the cases.

As to change in the micturation contraction pressure, little change was observed for both donepezil hydrochloride and vehicle administrations showing no significant difference (FIG. 13).

Thus, the examples above show that donepezil hydrochloride that activates ACh system selectively to brain activates preferentially over pons and thus ameliorates overactive bladder caused by cerebral infarction.

### INDUSTRIAL APPLICABILITY

The present invention provides a therapeutic agent for overactive bladder resulting from cerebral infarction comprising, as an active ingredient, a compound having a cholinesterase (ChE) inhibitory activity or a pharmacologically acceptable salt thereof. An overactive bladder therapeutic agent of the invention is useful as a novel therapeutic agent for urine collection disorder associated with overactive bladder resulting from cerebral infarction. The compound of the invention, for example, donepezil hydrochloride, has no side effects such as dry mouth, constipation and urinary excretion disorder which accompany the existing overactive bladder therapeutic agents. In addition, considering that most of the patients to be administered are elderly, donepezil hydrochloride can be administered safely without being concerned about damage in higher brain functions, and thus can be a innovative therapeutic agent for overactive bladder resulting from cerebral infarction.

What is claimed is:

1. A method for treating overactive bladder resulting from cerebral infarction, comprising administering a compound having a cholinesterase inhibitory activity, a pharmacologically acceptable salt or a solvate thereof to a patient with the overactive bladder resulting from cerebral infarction.

2. A method according to claim 1, wherein the compound having a cholinesterase inhibitory activity is a cyclic amine derivative represented by the following general formula: 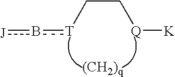
(wherein, J is:
(a) a substituted or unsubstituted (1) phenyl group, (2) pyridyl group, (3) pyradyl group, (4) quinolyl group, (5) cyclohexyl group, (6) quinoxalyl group or (7) furyl group;
(b) a monovalent or divalent group derived from a group selected from the group consisting of (1) indanyl, (2) indanonyl, (3) indenyl, (4) indenonyl, (5) indandionyl, (6) tetralonyl, (7) benzsuberonyl, (8) indanolyl, and (9) a group represented by formula 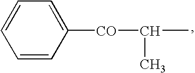
in all of which a phenyl group may be substituted;
(c) a monovalent group derived from a cyclic amide compound;
(d) a lower alkyl group; or
(e) a group represented by formula R¹—CH═CH— (wherein R¹ is a hydrogen atom or a lower alkoxycarbonyl group),
B is a group represented by formula 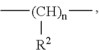
a group represented by formula 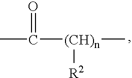
a group represented by formula 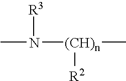
(wherein, R³ is a hydrogen atom, a lower alkyl group, an acyl group, a lower alkylsulfonyl group, a substituted or unsubstituted phenyl group or a benzyl group), a group represented by formula 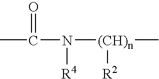
(wherein, R⁴ is a hydrogen atom, a lower alkyl group or a phenyl group), a group represented by formula 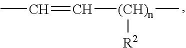
a group represented by formula 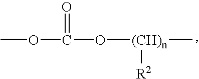
a group represented by formula 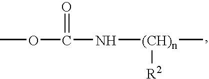
a group represented by formula 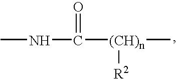
a group represented by formula 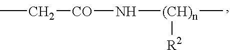
a group represented by formula 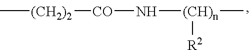
a group represented by formula 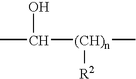
(wherein, n is 0 or an integer of 1 to 10, and R² is a hydrogen atom or a methyl group), a group represented by formula ═(CH—CH═CH)_{b}— (wherein, b is an integer of 1 to 3), a group represented by formula ═CH—(CH₂)_{c}— (wherein, c is 0 or an integer of 1 to 9), a group represented by formula ═(CH—CH)_{d}═ (wherein, d is 0 or an integer of 1 to 5), a group represented by formula 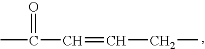
a group represented by formula 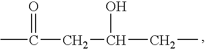
a group represented by formula 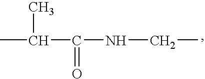
a group represented by formula 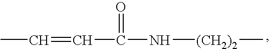
a group represented by formula —NH—, a group represented by formula —O—, a group represented by formula —S—, a dialkylaminoalkylcarbonyl group or a lower alkoxycarbonyl group,
T is a nitrogen atom or a carbon atom,
Q is a nitrogen atom, a carbon atom or a group represented by formula 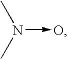
K is a hydrogen atom, a substituted or unsubstituted phenyl group, an arylalkyl group in which a phenyl group may be substituted, a cinnamyl group in which a phenyl group may be substituted, a lower alkyl group, a pyridylmethyl group, a cycloalkylalkyl group, an adamantanemethyl group, a furylmethyl group, a substituted or unsubstituted cycloalkyl group, a lower alkoxycarbonyl group or an acyl group,
q is an integer of 1 to 3, and [symbol]
indicates a single bond or a double bond).

3. A method according to claim 2, wherein J is a group selected from the group consisting of: substituted or unsubstituted (1) phenyl group, (2) pyridyl group, (3) pyradyl group, (4) quinolyl group, (5) cyclohexyl group, (6) quinoxalyl group and (7) furyl group.

4. A method according to claim 2, wherein J is a monovalent group derived from a cyclic amide compound.

5. A method according to claim 1, wherein a compound having a cholinesterase inhibitory activity is a cyclic amine derivatives represented by the following general formula: 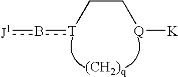
(wherein, J¹ is a monovalent or divalent group derived from a group selected from the group consisting of (1) indanyl, (2) indanonyl, (3) indenyl, (4) indenonyl, (5) indandionyl, (6) tetralonyl, (7) benzsuberonyl, (8) indanolyl, and (9) a group represented by formula 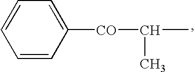
in all of which a phenyl group may be substituted,
B is a group represented by formula 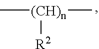
a group represented by formula 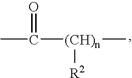
a group represented by formula 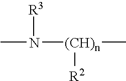
(wherein, R³ is a hydrogen atom, a lower alkyl group, an acyl group, a lower alkylsulfonyl group, a substituted or unsubstituted phenyl group or a benzyl group), a group represented by formula 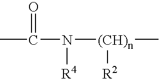
(wherein, R⁴ is a hydrogen atom, a lower alkyl group —CH═CH—(CH)ₙ— or a phenyl group), a group represented by formula 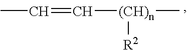
a group represented by formula 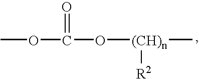
a group represented by formula 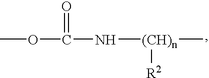
a group represented by formula 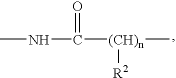
a group represented by formula 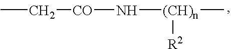
a group represented by formula 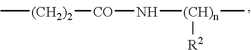
a group represented by formula 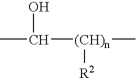
(wherein, n is 0 or an integer of 1 to 10, and R² is a hydrogen atom or a methyl group), a group represented by formula ═(CH—CH═CH)_{b}— (wherein, b is an integer of 1 to 3), a group represented by formula ═CH—(CH₂)_{c}— (wherein, c is 0 or an integer of 1 to 9), a group represented by formula ═(CH—CH)_{d}═ (wherein, d is 0 or an integer of 1 to 5), a group represented by formula 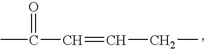
a group represented by formula 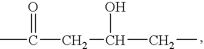
a group represented by formula 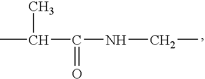
a group represented by formula 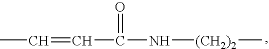
a group represented by formula —NH—, a group represented by formula —O—, a group represented by formula —S—, a dialkylaminoalkylcarbonyl group or a lower alkoxycarbonyl group,
T is a nitrogen atom or a carbon atom,
Q is a nitrogen atom, a carbon atom or a group represented by formula 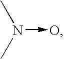
K is a hydrogen atom, a substituted or unsubstituted phenyl group, an arylalkyl group in which a phenyl group may be substituted, a cinnamyl group in which a phenyl group may be substituted, a lower alkyl group, a pyridylmethyl group, a cycloalkylalkyl group, an adamantanemethyl group, a furylmethyl group, a substituted or unsubstituted cycloalkyl group, a lower alkoxycarbonyl group or an acyl group,
q is an integer of 1 to 3, and [symbol]
indicates a single bond or a double bond).

6. A method according to claim 5, wherein B is a group represented by formula 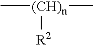
(wherein n is 0 or an integer of 1 to 10, and R² is a hydrogen atom or a methyl group), a group represented by formula —CH═CH—(CH)ₙR²— (wherein, n is 0 or an integer of 1 to 10, R² is a hydrogen atom or a methyl group), a group represented by formula ═(CH—CH═CH)_{b}— (wherein, b is an integer of 1 to 3), a group represented by formula ═CH—(CH₂)_{c}— (wherein, c is 0 or an integer of 1 to 9) or a group represented by formula ═(CH—CH)_{d}═ (wherein, d is 0 or an integer of 1 to 5).

7. A method according to claim 1, wherein a compound having a cholinesterase inhibitory activity is a cyclic amine derivative represented by the following general formula: 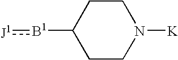
(wherein, J¹ is a monovalent or divalent group derived from a group selected from the group consisting of (1) indanyl, (2) indanonyl, (3) indenyl, (4) indenonyl, (5) indandionyl, (6) tetralonyl, (7) benzsuberonyl, (8) indanolyl, (9) a group represented by formula 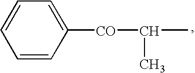
in all of which a phenyl group may be substituted,
B¹ is a group represented by formula 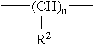
(wherein, n is 0 or an integer of 1 to 10, and R² is a hydrogen atom or a methyl group), a group represented by formula —CH═CH—(CH)ₙR²— (wherein, n is 0 or an integer of 1 to 10, and R² is a hydrogen atom or a methyl group), a group represented by formula ═(CH—CH═CH)_{b}— (wherein, b is an integer of 1 to 3), a group represented by formula ═CH—(CH₂)_{c}— (wherein, c is 0 or an integer of 1 to 9) or a group represented by formula ═(CH—CH)_{d}═ (wherein, d is 0 or an integer of 1 to 5), and
K is a hydrogen atom, a substituted or unsubstituted phenyl group, an arylalkyl group in which a phenyl group may be substituted, a cinnamyl group in which a phenyl group may be substituted, a lower alkyl group, a pyridylmethyl group, a cycloalkylalkyl group, an adamantanemethyl group, a furylmethyl group, a substituted or unsubstituted cycloalkyl group, a lower alkoxycarbonyl group or an acyl group).

8. A method according to claim 7, wherein K is a substituted or unsubstituted arylalkyl group or phenyl group.

9. A method according to either one of claims 7 and 8, wherein J¹ is a group selected from the group consisting of monovalent and divalent groups derived from indanonyl, indenyl and indandionyl.

10. A method according to either one of claims 7 and 8, wherein J¹ is an indanonyl group which may contain, as a substituent, a lower alkyl group with a carbon number 1 to 6 or a lower alkoxy group with a carbon number 1 to 6.

11. A method according to claim 2, wherein the cyclic amine derivative is at least one selected from the group consisting of: 1-benzyl-4-((5,6-dimethoxy-1-indanone)-2-yl)methylpiperidine, 1-benzyl-4-((5,6-dimethoxy-1-indanone)-2-ylidenyl)methylpiperidine, 1-benzyl-4-((5-methoxy-1-indanone)-2-yl)methylpiperidine, 1-benzyl-4-((5,6-methylenedioxy-1-indanone)-2-yl)methylpiperidine, 1-(m-nitrobenzyl)-4-((5,6-dimethoxy-1-indanone)-2-yl)methylpiperidine, 1-cyclohexylmethyl-4-((5,6-dimethoxy-1-indanone)-2-yl)methylpiperidine, 1-(m-fluorobenzyl)-4-((5,6-dimethoxy-1-indanone)-2-yl)methylpiperidine, 1-benzyl-4-(3-((5,6-dimethoxy-1-indanone)-2-yl)propyl)piperidine, 1-benzyl-4-((5-isopropoxy-6-methoxy-1-indanone)-2-yl)methylpiperidine, 1-benzyl-4-((5,6-dimethoxy-1-indanone)-2-ylidenyl)propenylpiperidine, and 1-benzyl-4-((5,6-dimethoxy-1,3-indandione)-2-yl)propenylpiperidine.

12. A method according to claim 2, wherein the cyclic amine derivative is 1-benzyl-4-((5,6-dimethoxy-1-indanone)-2-yl)methylpiperidine.

13. A method according to claim 1, wherein the compound having a cholinesterase inhibitory activity is 1-benzyl-4-((5,6-dimethoxy-1-indanone)-2-yl) methylpiperidine hydrochloride.

14. A method according to claim 1, wherein the compound having a cholinesterase inhibitory activity is galantamine, tacrine, physostigmine or rivastigmine.

15. A process for screening a substance for suppressing overactive bladder resulting from cerebral infarction, comprising: administering a compound having a cholinesterase inhibitory activity, a pharmacologically acceptable salt or a solvate thereof to a non-human mammal; and detecting or determining at least one selected from the group consisting of a bladder capacity, a bladder contraction pressure and an amount of retained urine, in the presence and absence of the compound, the pharmacologically acceptable salt or the solvate thereof.

16. A method according to claim 15, wherein the compound having a cholinesterase inhibitory activity is a compound having an acetylcholinesterase inhibitory activity, a pharmacologically acceptable salt or a solvate thereof.
